(19) United States
(12) Patent Application Publication
(10) Pub. No.: US 2022/0313701 A1
(43) Pub. Date: Oct. 6, 2022

(54) MODULATORS OF TREX1

(71) Applicant: Constellation Pharmaceuticals, Inc., Cambridge, MA (US)

(72) Inventors: Avinash Khanna, Cambridge, MA (US); Jonathan E. Wilson, Arlington, MA (US); Mary-Margaret Zablocki, Revere, MA (US)

(21) Appl. No.: 17/607,940

(22) PCT Filed: May 1, 2020
(86) PCT No.: PCT/US2020/030921

§ 371 Date: Nov. 1, 2021

Related U.S. Application Data
(60) Provisional application No. 62/842,149, filed on May 2, 2019.

(51) Int. Cl. A61K 31/5377; A61P 35/00

(52) U.S. Cl. CPC A61K 31/5377; A61P 35/00

(57) ABSTRACT

Provided are compounds of Formula (I): and pharmaceutically acceptable salts and compostions thereof, which are useful for treating a variety of conditions associated with TREX1.

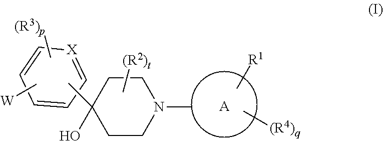

### RELATED APPLICATIONS

This application claims priority to U.S. Provisional Application No. 62/842,149 filed May 2, 2019, the entire contents of which are incorporated herein by reference.

### BACKGROUND

A potential immune therapy is needed for cancers related to the innate immune system recognition of non-self, and to detect and protect against potential danger. Cancer cells differ antigenically from their normal counterparts and emit danger signals to alert the immune system similar to viral infection. These signals, which include damage-associated molecular patterns (DAMPs) and pathogen-associated molecular patterns (PAMPs), further activate the innate immune system resulting in the protection of the host from a variety of threats (Front. Cell Infect. Microbiol. 2012, 2, 168).

Ectopically expressed single stranded DNA (ssDNA) and double stranded DNA (dsDNA) are known PAMPs and/or DAMPs, which are being recognized by the cyclic GMP-AMP synthase (cGAS), a nucleic acid sensor (Nature 2011, 478, 515-518). Upon sensing of cytosolic DNA, cGAS catalyzes the generation of the cyclic dinucleotide 2′,3′-cGAMP, a potent second messenger and activator of the ER transmembrane adapter protein stimulator of interferon genes (STING) (Cell Rep. 2013, 3, 1355-1361). STING activation triggers phosphorylation of IRF3 via TBK1 which in turn leads to type I interferon production and activation of interferon stimulated genes (ISGs); a pre-requisite to the activation of innate immunity and initiation of adaptive immunity. Production of type I interferons thus constitutes a key bridge between the innate and adaptive immunity (Science 2013, 341, 903-906).

Excess type I IFN can be harmful to the host and induce autoimmunity, therefore, negative feedback mechanisms exist that keep type I IFN-mediated immune activation in check. Three prime repair exonuclease I (TREX1) is a 3′-5′ DNA exonuclease responsible for the removal of ectopically expressed ssDNA and dsDNA and is therefore a key repressor of the cGAS/STING pathway (PNAS 2015, 112, 5117-5122).

Type I interferons and downstream pro-inflammatory cytokine responses are critical to the development of immune responses and their effectiveness. Type I interferons enhance both the ability of dendritic cells and macrophages to take up, process, present, and cross-present antigens to T cells, and their potency to stimulate T cells by eliciting the up-regulation of the co-stimulatory molecules such as CD40, CD80 and CD86 (J. Exp. Med. 2011, 208, 2005-2016). Type I interferons also bind their own receptors and activate interferon responsive genes that contribute to activation of cells involved in adaptive immunity (EMBO Rep. 2015, 16, 202-212).

From a therapeutic perspective, type I interferons and compounds that can induce type I interferon production have potential for use in the treatment of human cancers (Nat. Rev Immunol. 2015, 15, 405-414). Interferons can inhibit human tumor cell proliferation directly. In addition, type I interferons can enhance anti-tumor immunity by triggering the activation of cells from both the innate and adaptive immune system. Importantly, the anti-tumor activity of PD-1 blockade requires pre-existing intratumoral T cells. By turning cold tumors into hot and thereby eliciting a spontaneous anti-tumor immunity, type I IFN-inducing therapies have the potential to expand the pool of patients responding to anti-PD-1 therapy as well as enhance the effectiveness of anti-PD1 therapy.

Therapies that are currently in development that induce a potent type I interferon response require focal or intratumoral administration to achieve an acceptable therapeutic index. Thus, there remains a need for new agents with systemic delivery and lower toxicity to expand the benefit of type I IFN-inducing therapies to patients without peripherally treatment accessible lesions. Human and mouse genetic studies suggest that TREX1 inhibition might be amenable to a systemic delivery route and therefore TREX1 inhibitory compounds could play an important role in the anti-tumor therapy landscape. TREX1 is a key determinant for the limited immunogenicity of cancer cells responding to radiation treatment [Trends in Cell Biol., 2017, 27 (8), 543-4; Nature Commun., 2017, 8, 15618]. TREX1 is induced by genotoxic stress and involved in protection of glioma and melanoma cells to anticancer drugs [Biochim. Biophys. Acta, 2013, 1833, 1832-43]. STACT-TREX1 therapy shows robust anti-tumor efficacy in multiple murine cancer models [Glickman et al, Poster P235, 33^{rd} Annual Meeting of Society for Immunotherapy of Cancer, Washington D.C., November 7-11, 2018].

### SUMMARY

Provided herein are compounds having the Formula I:

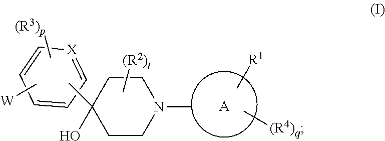

and pharmaceutically acceptable salts and compositions thereof, wherein R¹, R², R³, W, q, p, and t are as described herein. The disclosed compounds and compositions are useful in modulating modulate TREX1, and are useful in a variety of therapeutic applications such as, for example, in treating cancer.

### BRIEF DESCRIPTION OF THE FIGURES

FIG. 1A illustrates the results from a knock down experiment of TREX1 in B16F10 tumor cells using CRISPR. FIG. 1B illustrates TREX1 attenuated the activation of the cGAS/STING pathway in B16F10 tumor cells.

FIG. 2. illustrates that tumors in which TREX had been silenced had smaller volumes compared with parental B16F10 tumors.

FIG. 3. shows that TREX1 knockout B16F10 tumors exhibited a significant increase in overall immune cells. This reflected an increase in the number of tumor infiltrating CD4 and CD8 T cells as well as in plasmacytoid dendritic cells (pDCs).

### DETAILED DESCRIPTION

### 1. General Description of Compounds

In a first embodiment, provided herein is a compound of Formula I:

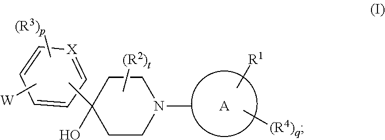

or a pharmaceutically acceptable salt thereof, wherein:

W is fluoro substituted meta or para to the piperidine;

X is independently N or C;

Ring A is a 5-membered heteroaryl or a 6-membered heteroaryl, wherein said 6-membered heteroaryl is substituted meta to the piperidine by R¹;

R¹ is phenyl, heteroaryl, heterocyclyl, C₁-C₆ alkoxy, C₁-C₆ alkyl, C₁-C₆ haloalkyl, hydroxyC₁-C₆ alkyl, —C(O)NR^{a}R^{b}, —NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OC₁-C₆ alkyl, or —SC₁-C₆ alkyl, wherein each of said phenyl, heteroaryl, and heterocyclyl are optionally substituted with 1 to 4 groups selected from R⁶;

R² is halo, hydroxy, C₁-C₆ alkyl, C₁-C₆ haloalkyl, hydroxyC₁-C₆ alkyl, C₁-C₆ alkoxy, C₁-C₆ haloalkoxy, —C(O)NR^{a}R^{b}, —NR^{a}R^{b}, —COOR^{c}, —SO₂R^{c}, —NR^{a}C(O)OR^{c}, —NR^{a}C(S)OR^{c}, or —NR^{a}C(O)R^{c};

each R³, if present, is independently halo, hydroxy, C₁-C₆ alkyl, C₁-C₆ haloalkyl, hydroxyC₁-C₆ alkyl, C₁-C₆ alkoxy, or C₁-C₆ haloalkoxy;

R⁴ is heteroaryl, halo, C₁-C₆ alkoxy, C₁-C₆ alkyl, C₁-C₆ haloalkyl, hydroxyC₁-C₆ alkyl, oxo, —C(O)NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OR^{c}, or —SR^{c}, wherein said heteroaryl is optionally substituted with 1 to 3 groups selected from R⁵;

R⁵ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c}

R⁶ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c};

each R^{a} is independently hydrogen or C₁-C₆ alkyl;

each R^{b} is independently hydrogen or C₁-C₆ alkyl optionally substituted with 1 or 2 groups selected from phenyl, heteroaryl, OR^{c}, and —NR^{c}R^{d}; or R^{a} and R^{b} together with the nitrogen atom to which they are attached form a nitrogen containing heterocyclyl optionally substituted with 1 to 4 groups selected from halo, C₁-C₆ alkyl, C₁-C₆ haloalkyl, C₁-C₆ alkoxy, and C₁-C₆ haloalkoxy;

each R^{c} and R^{d} are independently hydrogen or C₁-C₆ alkyl;

p is 0, 1, or 2;

t is 0, 1, or 2; and

q is 0, 1, or 2;

provided the compound of Formula I is not 1-(2-amino-6-methylpyrimidin-4-yl)-4-(4-fluorophenyl)piperidin-4-ol, (R)-4-(4-fluorophenyl)-1-(6-((2-hydroxy-2-phenylethyl)amino)pyrimidin-4-yl)piperidin-4-ol, 4-(4-fluorophenyl)-1-(4-(1,3,5-trimethyl-1H-pyrazol-4-yl)pyrimidin-2-yl)piperidin-4-ol, 4-(4-fluorophenyl)-1-(2-methyl-6-(piperidin-3-yl)pyrimidin-4-yl)piperidin-4-ol, 4-(4-fluorophenyl)-1-(2,5,6-trimethylpyrimidin-4-yl)piperidin-4-ol, 1-(2-amino-5-ethylpyrimidin-4-yl)-4-(4-fluorophenyl)piperidin-4-ol, 4-(4-fluorophenyl)-1-(4-methylpyrimidin-2-yl)piperidin-4-ol, 4-(4-fluorophenyl)-1-(4-(pyridin-3-yl)pyrimidin-2-yl)piperidin-4-ol, 4-(4-fluorophenyl)-1-(4-(pyridin-2-yl)pyrimidin-2-yl)piperidin-4-ol, 4-(4-fluorophenyl)-1-(4-(pyridin-2-yl)pyrimidin-2-yl)piperidin-4-ol, 4-(4-fluorophenyl)-1-(4-methoxy-6-methylpyrimidin-2-yl)piperidin-4-ol, or 4-(4-fluorophenyl)-1-(4-methyl-6-morpholinopyrimidin-2-yl)piperidin-4-ol, or a pharmaceutically acceptable salt of any of the foregoing.

In a second embodiment, provided herein is a pharmaceutical composition comprising 1) a compound of Formula I:

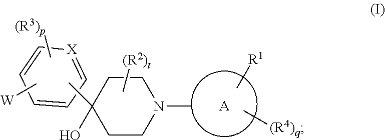

or a pharmaceutically acceptable salt thereof, wherein:

W is fluoro substituted meta or para to the piperidine;

X is independently N or C;

Ring A is a 5-membered heteroaryl or a 6-membered heteroaryl, wherein said 6-membered heteroaryl is substituted meta to the piperidine by R¹;

R¹ is phenyl, heteroaryl, heterocyclyl, C₁-C₆ alkoxy, C₁-C₆ alkyl, C₁-C₆ haloalkyl, hydroxyC₁-C₆ alkyl, —C(O)NR^{a}R^{b}, —NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OC₁-C₆ alkyl, or —SC₁-C₆ alkyl, wherein each of said phenyl, heteroaryl, and heterocyclyl are optionally substituted with 1 to 4 groups selected from R⁶.

R² is halo, hydroxy, C₁-C₆ alkyl, C₁-C₆ haloalkyl, hydroxyC₁-C₆ alkyl, C₁-C₆ alkoxy, C₁-C₆ haloalkoxy, —C(O)NR^{a}R^{b}, —NR^{a}R^{b}, —COOR^{c}, —SO₂R^{c}, —NR^{a}C(O)OR^{c}, —NR^{a}C(S)OR^{c}, or —NR^{a}C(O)R^{c};

each R³, if present, is independently halo, hydroxy, C₁-C₆ alkyl, C₁-C₆ haloalkyl, hydroxyC₁-C₆ alkyl, C₁-C₆ alkoxy, or C₁-C₆ haloalkoxy;

R⁴ is heteroaryl, halo, C₁-C₆ alkoxy, C₁-C₆ alkyl, C₁-C₆ haloalkyl, hydroxyC₁-C₆ alkyl, oxo, —C(O)NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OR^{c}, or —SR^{c}, wherein said heteroaryl is optionally substituted with 1 to 3 groups selected from R⁵;

R⁵ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c}

R⁶ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c};

each R^{a} is independently hydrogen or C₁-C₆ alkyl;

each R^{b} is independently hydrogen or C₁-C₆ alkyl optionally substituted with 1 or 2 groups selected from phenyl, heteroaryl, OR^{c}, and —NR^{c}R^{d}; or R^{a} and R^{b} together with the nitrogen atom to which they are attached form a nitrogen containing heterocyclyl optionally substituted with 1 to 4 groups selected from halo, C₁-C₆ alkyl, C₁-C₆ haloalkyl, C₁-C₆ alkoxy, and C₁-C₆ haloalkoxy;

each R^{c} and R^{d} are independently hydrogen or C₁-C₆ alkyl;

p is 0, 1, or 2;

t is 0, 1, or 2; and

q is 0, 1, or 2; and

2) a pharmaceutically acceptable carrier.

### 2. Definitions

When used in connection to describe a chemical group that may have multiple points of attachment, a hyphen (-) designates the point of attachment of that group to the variable to which it is defined. For example, —NHC(O)OR^{a} and —NHC(S)OR^{a} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical. Unless otherwise specified, an alkyl group typically has 1-4 carbon atoms, i.e., (C₁-C₄)alkyl.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., but are not limited to —OCHCF₂ or —OCF₃.

The term “heteroaryl” used alone or as part of a larger moiety refers to a 5- to 12-membered (e.g., a 5- to 7-membered or 5- to 6-membered) aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered (e.g., a 4- to 7-membered or 4- to 6-membered) saturated or partially unsaturated heterocyclic ring containing 1 to 4 heteroatoms independently selected from N, O, and S. It can be mononcyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “spiro” refers to two rings that shares one ring atom (e.g., carbon).

The term “fused” refers to two rings that share two adjacent ring atoms with one another.

The term “bridged” refers to two rings that share three ring atoms with one another.

The term “TREX1” refers to Three prime repair exonuclease 1 or DNA repair exonuclease 1, which is an enzyme that in humans is encoded by the TREX1 gene. Mazur D J, Perrino F W (August 1999). “Identification and expression of the TREX1 and TREX2 cDNA sequences encoding mammalian 3′-->5′ exonucleases”. J Biol Chem. 274 (28): 19655-60. doi:10.1074/jbc.274.28.19655. PMID 10391904; Hoss M, Robins P, Naven T J, Pappin D J, Sgouros J, Lindahl T (August 1999). “A human DNA editing enzyme homologous to the Escherichia coli DnaQ/MutD protein”. EMBO J. 18 (13): 3868-75. doi:10.1093/emboj/18.13.3868. PMC 1171463. PMID 10393201. This gene encodes the major 3′->5′ DNA exonuclease in human cells. The protein is a non-processive exonuclease that may serve a proofreading function for a human DNA polymerase. It is also a component of the SET complex, and acts to rapidly degrade 3′ ends of nicked DNA during granzyme A-mediated cell death. Cells lacking functional TREX1 show chronic DNA damage checkpoint activation and extra-nuclear accumulation of an endogenous single-strand DNA substrate. It appears that TREX1 protein normally acts on a single-stranded DNA polynucleotide species generated from processing aberrant replication intermediates. This action of TREX1 attenuates DNA damage checkpoint signaling and prevents pathological immune activation. TREX1 metabolizes reverse-transcribed single-stranded DNA of endogenous retroelements as a function of cell-intrinsic antiviral surveillance, resulting in a potent type I IFN response. TREX1 helps HIV-1 to evade cytosolic sensing by degrading viral cDNA in the cytoplasm.

The term “TREX2” refers to Three prime repair exonuclease 2 is an enzyme that in humans is encoded by the TREX2 gene. This gene encodes a nuclear protein with 3′ to 5′ exonuclease activity. The encoded protein participates in double-stranded DNA break repair, and may interact with DNA polymerase delta. Enzymes with this activity are involved in DNA replication, repair, and recombination. TREX2 is a 3′-exonuclease which is predominantly expressed in keratinocytes and contributes to the epidermal response to UVB-induced DNA damage. TREX2 biochemical and structural properties are similar to TREX1, although they are not identical. The two proteins share a dimeric structure and can process ssDNA and dsDNA substrates in vitro with almost identical k_{cat} values. However, several features related to enzyme kinetics, structural domains, and subcellular distribution distinguish TREX2 from TREX1. TREX2 present a 10-fold lower affinity for DNA substrates in vitro compared with TREX1. In contrast with TREX1, TREX2 lacks a COOH-terminal domain that can mediate protein-protein interactions. TREX2 is localized in both the cytoplasm and nucleus, whereas TREX1 is found in the endoplasmic reticulum, and is mobilized to the nucleus during granzyme A-mediated cell death or after DNA damage.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a third embodiment, p in the compound of Formula I is 1 or 2, wherein the remaining variables are as described above for Formula I in the first or second embodiment.

In a fourth embodiment, the compound of Formula I is of the Formula II:

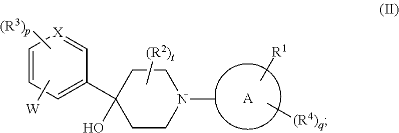

or a pharmaceutically acceptable salt thereof, wherein the variables are as described above in the first, second, or third embodiment.

In a fifth embodiment, the compound of Formula I is of the Formula III:

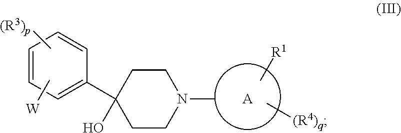

or a pharmaceutically acceptable salt thereof, wherein the variables are as described above in the first, second, or third embodiment.

In a sixth embodiment, the compound of Formula I is of the Formula IV:

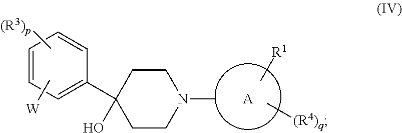

or a pharmaceutically acceptable salt thereof, wherein the variables are as described above in the first, second, or third embodiment.

In a seventh embodiment, p in the compound of Formula I, II, III, or IV is 1, wherein the remaining variables are as described above in the first, second, or third embodiment.

In an eighth embodiment, R³ in the compound of Formula I, II, III, or IV is halo or C₁-C₆ alkyl, wherein the remaining variables are as described above in the first, second, third, or seventh embodiment. Alternatively, as part of an eight embodiment, R³ in the compound of Formula I, II, III, or IV is methyl, fluoro, or chloro, wherein the remaining variables are as described above in the first, second, third, or seventh embodiment.

In a ninth embodiment, Ring A in the compound of Formula I, II, III, or IV is selected from:

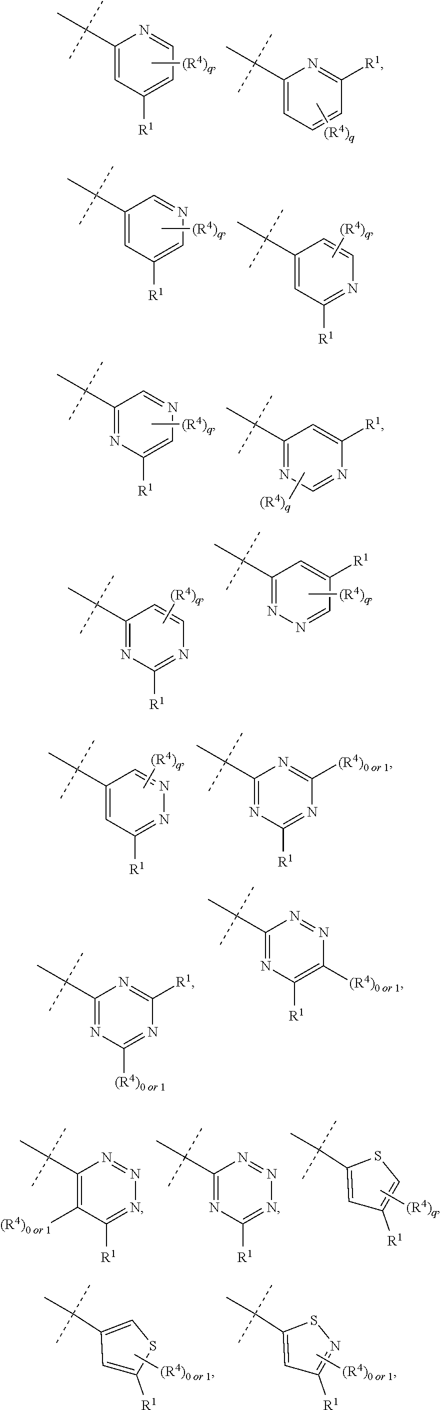

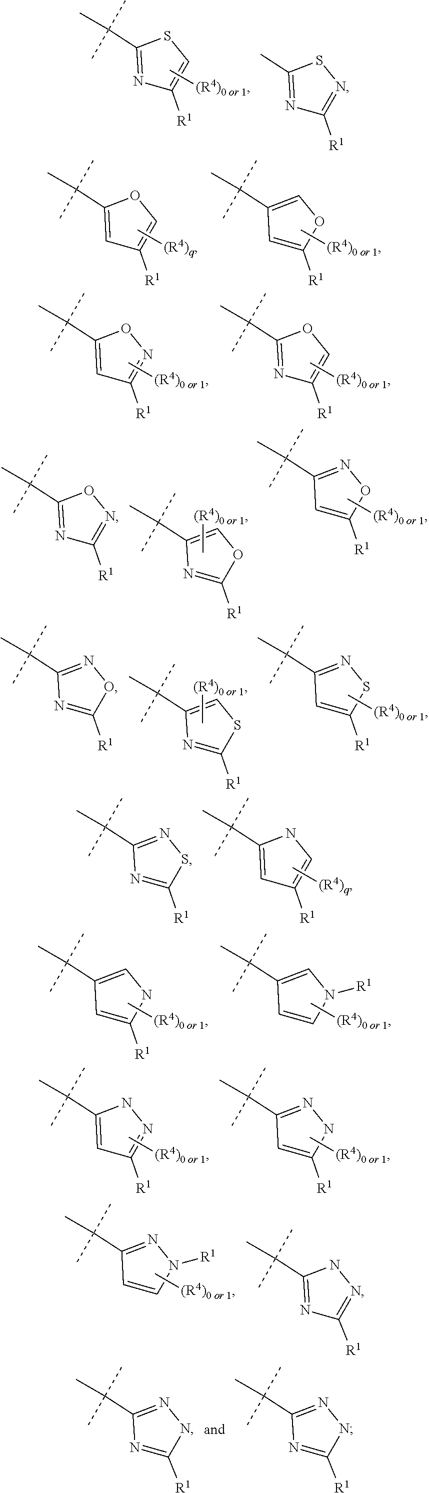

and

R¹ is phenyl, heteroaryl, heterocyclyl, C₁-C₆ alkoxy, C₁-C₆ alkyl, C₁-C₆ haloalkyl, hydroxyC₁-C₆ alkyl, —C(O)NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OC₁-C₆ alkyl, or —SC₁-C₆ alkyl, wherein each of said phenyl, heteroaryl, and heterocyclyl are optionally substituted with 1 to 2 groups selected from R⁶ and wherein the remaining variables are as described above in the first, second, third, seventh, or eighth embodiment. Alternatively, as part of a ninth embodiment, Ring A in the compound of Formula I, II, III, or IV is

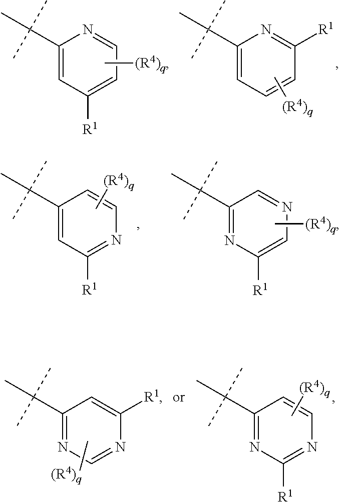

wherein the remaining variables are as described above in the first, second, third, seventh, or eighth embodiment. In one aspect of the ninth embodiment, the compound is not 4-(4-fluorophenyl)-1-(2-methyl-6-(piperidin-3-yl)pyrimidin-4-yl)piperidin-4-ol.

In a tenth embodiment, q in the compound of Formula I, II, III, or IV is 0 or 1, wherein the remaining variables are as described above in the first, second, third, seventh, eighth, or ninth embodiment.

In an eleventh embodiment, R⁴ in the compound of Formula I, II, III, or IV is 5- to 6-membered heteroaryl, —COOR^{c}, —C(O)NR^{a}R^{b}, C₁-C₆ alkyl, C₁-C₆ haloalkyl, hydroxyC₁-C₆ alkyl, wherein said 5- to 6-membered heteroaryl is optionally substituted with 1 or 2 groups selected from R⁵, wherein the remaining variables are as described above in the first, second, third, seventh, eighth, ninth, or tenth embodiment. Alternatively, as part of an eleventh embodiment, R⁴ in the compound of Formula I, II, III, or IV is pyrazolyl, —COOR^{c}, —C(O)NR^{a}R^{b}, C₁-C₄ alkyl, C₁-C₄ haloalkyl, hydroxyC₁-C₄ alkyl, wherein said pyrazolyl is optionally substituted with 1 or 2 groups selected from R⁵, wherein the remaining variables are as described above in the first, second, third, seventh, eighth, ninth, or tenth embodiment.

In a twelfth embodiment, R⁵ in the compound of Formula I, II, III, or IV is C₁-C₄ alkyl, wherein the remaining variables are as described above in the first, second, third, seventh, eighth, ninth, tenth, or eleventh embodiment.

In a thirteenth embodiment, R¹ in the compound of Formula I, II, III, or IV is phenyl, heteroaryl, heterocyclyl, halo, C₁-C₆ alkoxy, C₁-C₆ alkyl, C₁-C₆ haloalkyl, hydroxyC₁-C₆ alkyl, —C(O)NR^{a}R^{b}, or —COOR^{c}, wherein each of said phenyl, heteroaryl, and heterocyclyl are optionally substituted with 1 to 3 groups selected from R⁶, wherein the remaining variables are as described above in the first, second, third, seventh, eighth, ninth, tenth, eleventh, or twelfth embodiment. Alternatively, as part of a thirteenth embodiment, R¹ in the compound of Formula I, II, III, or IV is phenyl, 5- to 6-membered nitrogen containing heteroaryl, 5- to 6-membered nitrogen containing heterocyclyl, halo, C₁-C₃ alkoxy, C₁-C₃ alkyl, C₁-C₃ haloalkyl, oxo, —C(O)NR^{a}R^{b}, or —COOR^{c}, wherein each of said phenyl, heteroaryl, and heterocyclyl are optionally substituted with 1 to 3 groups selected from R⁶, wherein the remaining variables are as described above in the first, second, third, seventh, eighth, ninth, tenth, eleventh, or twelfth embodiment. In another alternative, as part of a thirteenth embodiment, R¹ in the compound of Formula I, II, III, or IV is Cl, OCH₃, CH₃, CF₃, —C(CH₃)₂OR^{c}, —CH₂OR^{c}, CF₃, oxo, —COOR^{c}, or —C(O)NR^{a}R^{b}, phenyl, pyrazolyl, imidazolyl, isoxazolyl, triazolyl, pyridinyl, pyrimidinyl, or pyrrolidinyl, wherein each of said phenyl, pyrazolyl, imidazolyl, isoxazolyl, triazolyl, pyridinyl, pyrimidinyl, or pyrrolidinyl is optionally substituted with 1 to 3 groups selected from R⁶, wherein the remaining variables are as described above in the first, second, third, seventh, eighth, ninth, tenth, eleventh, or twelfth embodiment.

In a fourteenth embodiment, R⁶ in the compound of Formula I, II, III, or IV is selected from halo, C₁-C₆ alkyl, C₁-C₆ haloalkyl, C₁-C₆ alkoxy, cycloalkyl, cyano, —C(O)NR^{a}R^{b}, and —SO₂R^{c}, wherein said C₁-C₆ alkyl is optionally substituted with phenyl, wherein the remaining variables are as described above in the first, second, third, seventh, eighth, ninth, tenth, eleventh, twelfth, or thirteenth embodiment. Alternatively, as part of a fourteenth embodiment, R⁶ in the compound of Formula I, II, III, or IV is selected from halo, C₁-C₃ alkyl, C₁-C₃ haloalkyl, C₁-C₃ alkoxy, 3- to 5-membered monocyclic cycloalkyl, cyano, —C(O)NR^{a}R^{b}, and —SO₂R^{c}, wherein said C₁-C₃ alkyl is optionally substituted with phenyl, wherein the remaining variables are as described above in the first, second, third, seventh, eighth, ninth, tenth, eleventh, twelfth, or thirteenth embodiment. In another alternative, as part of a fourteenth embodiment, R⁶ in the compound of Formula I, II, III, or IV is selected from F, CH₃, CF₃, CHF₂, OCH₃, cyclopropyl, cyano, benzyl, —C(O)NR^{a}R^{b}, or —SO₂R^{c}, wherein the remaining variables are as described above in the first, second, third, seventh, eighth, ninth, tenth, eleventh, twelfth, or thirteenth embodiment.

In a fifteenth embodiment, each R^{a} in the compound of Formula I, II, III, or IV is independently hydrogen or CH₃, wherein the remaining variables are as described above in the first, second, third, seventh, eighth, ninth, tenth, eleventh, twelfth, thirteenth, or fourteenth embodiment.

In a sixteenth embodiment, each R^{b} in the compound of Formula I, II, III, or IV is independently hydrogen or C₁-C₆ alkyl optionally substituted with 1 or 2 groups selected from phenyl, nitrogen containing heteroaryl, OR^{c}, or —NR^{c}R^{d}; or R^{a} and R^{b} together with the nitrogen atom to which they are attached form a nitrogen containing heterocyclyl optionally substituted with C₁-C₆ alkyl, wherein the remaining variables are as described above in the first, second, third, seventh, eighth, ninth, tenth, eleventh, twelfth, thirteenth, fourteenth, or fifteenth embodiment. Alternatively, as part of a sixteenth embodiment, each Re in the compound of Formula I, II, III, or IV is independently hydrogen or C₁-C₃ alkyl optionally substituted with 1 or 2 groups selected from phenyl, 5- or 6-membered nitrogen containing heteroaryl, OR^{c}, or —NR^{c}R^{d}; or R^{a} and R^{b} together with the nitrogen atom to which they are attached form a 5- or 6-membered nitrogen containing heterocyclyl optionally substituted with C₁-C₃ alkyl, wherein the remaining variables are as described above in the first, second, third, seventh, eighth, ninth, tenth, eleventh, twelfth, thirteenth, fourteenth, or fifteenth embodiment. In another alternative, as part of a sixteenth embodiment, each Re in the compound of Formula I, II, III, or IV is independently hydrogen or C₁-C₃ alkyl optionally substituted with 1 or 2 groups selected from phenyl, pyridinyl, OR^{c}, or —NR^{c}R^{d}; or R^{a} and Re together with the nitrogen atom to which they are attached form a piperidinyl or piperazinyl optionally substituted with C₁-C₃ alkyl, wherein the remaining variables are as described above in the first, second, third, seventh, eighth, ninth, tenth, eleventh, twelfth, thirteenth, fourteenth, or fifteenth embodiment.

In an eighteenth embodiment, each R^{c} and R^{d} in the compound of Formula I, II, III, or IV are independently hydrogen or CH₃, wherein the remaining variables are as described above in the first, second, third, seventh, eighth, ninth, tenth, eleventh, twelfth, thirteenth, fourteenth, fifteenth, or sixteenth embodiment.

Specific examples of compounds are provided in the EXEMPLIFICATION section and are included as part of a seventeenth embodiment herein. Pharmaceutically acceptable salts as well as the neutral forms of these compounds are also included.

### 4. Uses, Formulation and Administration

Compounds and compositions described herein are generally useful for modulating the activity of TREX1. In some aspects, the compounds and pharmaceutical compositions described herein inhibit the activity TREX1.

In some aspects, compounds and pharmaceutical compositions described herein are useful in treating a disorder associated with TREX1 function. Thus, provided herein are methods of treating a disorder associated with TREX1 function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with TREX1 function. Also provided is a compound described herein, or a pharmaceutically acceptable salt thereof, or a pharmaceutical composition comprising a disclosed compound or pharmaceutically acceptable salt thereof, for use in treating a disorder associated with TREX1.

In some aspects, the compounds and pharmaceutical compositions described herein are useful in treating cancer.

In some aspects, the cancer treated by the compounds and pharmaceutical compositions described herein is selected from colon cancer, gastric cancer, thyroid cancer, lung cancer, leukemia, pancreatic cancer, melanoma, multiple melanoma, brain cancer, CNS cancer, renal cancer, prostate cancer, ovarian cancer, leukemia, and breast cancer.

In some aspects, the cancer treated by the compounds and pharmaceutical compositions described herein is selected from lung cancer, breast cancer, pancreatic cancer, colorectal cancer, and melanoma.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

Representative examples of the disclosed compounds are illustrated in the following non-limiting methods, schemes, and examples.

General starting materials used were obtained from commercial sources or prepared in other examples, unless otherwise noted.

The following abbreviations have the indicated meanings:

Ac=acetyl; ACN=acetonitrile; AcO acetate; BOC=t-butyloxycarbonyl; CBZ=carbobenzoxy; CDI=carbonyldiimidazole; DBU=1,8-Diazabicycloundec-7-ene; DCC=1,3-dicyclohexylcarbodiimide; DCE=1,2-dichloroethane; DI=de-ionized; DIAD=Diisopropyl azodicarboxylate; DIBAL=diisobutyl aluminum hydride; DIPA=diisopropylamine; DIPEA or DIEA=N,N-diisoproylethylamine, also known as Hunig's base; DMA=dimethylacetamide; DMAP=4-(dimethylamino)pyridine; DMF=dimethylformamide; DMP=Dess-Martin periodinane; DPPA=Diphenylphosphoryl azide; DPPP=1,3-bis(diphenylphosphino)propane; Dtbbpy=4,4′-di-/e/7-butyl-2,2′-dipyridyl; EDC or EDCI=1-(3-dimethylaminopropyl)-3-ethylcarbodiimide hydrochloride; EDTA=ethylenediaminetetraacetic acid, tetrasodium salt; EtOAc=ethyl acetate; FAB=fast atom bombardment; FMOC=9-fluorenylmethoxycarbonyl; HMPA=hexamethylphosphoramide; HATU=(9-(7-Azabenzotriazol-1-yl)-N, N, N, N-tetramethyluroniumhexafluorophosphate; HOAt=1-Hydroxy-7-azabenzotriazole or 3H-[1,2,3]triazolo[4,5-b]pyridin-3-ol; HOBt=1-hydroxybenzotriazole; HRMS=high resolution mass spectrometry; KHMDS=potassium hexamethyldisilazane; LC-MS=Liquid chromatography-mass spectrometry; LDA=lithium diisopropylamide; LiHMDS=lithium hexamethyldisilazane; MCPBA=meta-chloroperbenzoic acid; MMPP=magnesium monoperoxyphthlate hexahydrate; Ms=methanesulfonyl=mesyl; MsO=methanefulfonate=mesylate; MTBE=Methyl t-butyl ether; NBS=N-bromosuccinimide; NMM=4-methylmorpholine; NMP=N-methylpyrrolidinone; NMR=Nuclear magnetic resonance; PCC=pyridinium chlorochromate; PDC=pyridinium dichromate; Ph=phenyl; PPTS=pyridiniump-toluene sulfonate; pTSA=p-toluene sulfonic acid; r.t./RT=room temperature; rac.=racemic; T3P=2,4,6-Tripropyl-1,3,5,2,4,6-trioxatriphosphinane 2,4,6-trioxide; TEA=triethylamine; TFA=trifluoroacetic acid; TfO=trifluoromethanesulfonate=triflate; THF=tetrahydrofuran; TLC=thin layer chromatography; TMSCl=trimethylsilyl chloride.

The progress of reactions was often monitored by TLC or LC-MS. The LC-MS was recorded using one of the following methods.

### LCMS Method-1

| Mobile Phase | (A) | 2 mM Ammonium acetate + 0.1% | | |
|---|---|---|---|---|
| | | Formic Acid in Water | | |
| | (B) | 0.1% Formic Acid in Acetonitrile | | |
| Column: | | BEH C18 (50*2.1 mm) 1.7 um | | |
| Column Flow: | | 0.55 ml/min | | |
| Gradient: | | Time (min) | % A | % B |
| | | 0.01 | 98 | 2 |
| | | 0.30 | 98 | 2 |
| | | 0.60 | 50 | 50 |
| | | 1.10 | 25 | 75 |
| | | 2.00 | 0 | 100 |
| | | 2.70 | 0 | 100 |
| | | 2.71 | 98 | 2 |
| | | 3.00 | 98 | 2 |

### LCMS Method-2

| Mobile Phase | (A) | 5 mM Ammonium Acetate + 0.1% | | |
|---|---|---|---|---|
| | | Formic Acid in Water | | |
| | (B) | 0.1% Formic Acid in Acetonitrile | | |
| Column: | | BEH C18 (50*2.1 mm), 1.7 um or Equivalent | | |
| Column Flow: | | 0.45 ml/min | | |
| Gradient: | | Time (min) | % A | % B |
| | | 0.01 | 98 | 2 |
| | | 0.50 | 98 | 2 |
| | | 5.00 | 10 | 90 |
| | | 6.00 | 5 | 95 |
| | | 7.00 | 5 | 95 |
| | | 7.01 | 98 | 2 |
| | | 8.00 | 98 | 2 |

### LCMS Method-3

| Mobile Phase | (A) | 5 mM Ammonium bicarbonate in water | | |
|---|---|---|---|---|
| | (B) | Acetonitrile | | |
| Column: | | X-Bridge C18 (50*4.6 mm), 3.5 um | | |
| Column Flow: | | 1.0 ml/min | | |
| Gradient: | | Time (min) | % A | % B |
| | | 0.01 | 95 | 5 |
| | | 5.00 | 10 | 90 |
| | | 5.80 | 5 | 95 |
| | | 7.20 | 5 | 95 |
| | | 7.21 | 95 | 5 |
| | | 10.00 | 95 | 5 |

### LCMS Method-4

| Mobile Phase | (A) | 10 mM Ammonium Acetate in WATER | | |
|---|---|---|---|---|
| | (B) | 100% Acetonitrile | | |
| Column: | | X-Bridge C18 (150*4.6 mm), 5 um or Equivalent | | |
| Column Flow: | | 1.0 ml/min | | |
| Gradient: | | Time (min) | % A | % B |
| | | 0.01 | 90 | 10 |
| | | 5.00 | 10 | 90 |
| | | 7.00 | 0 | 100 |
| | | 11.00 | 0 | 100 |
| | | 11.01 | 90 | 10 |
| | | 12.00 | 90 | 10 |

### LCMS Method-5

| Mobile Phase | (A) | 10 mM Ammonium Acetate in Water | | |
|---|---|---|---|---|
| | (B) | 100% Acetonitrile | | |
| Column: | | X-Bridge C18 (150*4.6 mm), 5 um or Equivalent | | |
| Column Flow: | | 1.0 ml/min | | |
| Gradient: | | Time (min) | % A | % B |
| | | 0.01 | 100 | 0 |
| | | 7.00 | 50 | 50 |
| | | 9.00 | 0 | 100 |
| | | 11.00 | 0 | 100 |
| | | 11.01 | 100 | 0 |
| | | 12.00 | 100 | 0 |

### LCMS Method-5

| Mobile Phase | (A) | 0.1% Formic Acid in Water | | |
|---|---|---|---|---|
| | (B) | 0.1% Formic Acid in Acetonitrile | | |
| Column: | | Zorbax SB-C8 (4.5 × 75 mm), 3.5 μm | | |
| Column Flow: | | 1.5 ml/min | | |
| Gradient: | | Time (min) | % A | % B |
| | | 0.00 | 95 | 5 |
| | | 3.60 | 5 | 95 |
| | | 4.00 | 5 | 95 |
| | | 4.50 | 95 | 5 |

NMR was recorded at room temperature unless noted otherwise on Varian Inova 400 or 500 MHz spectrometers with the solvent peak used as the reference or on Bruker 300 or 400 MHz spectrometers with the TMS peak used as internal reference.

The compounds described herein may be prepared using the following methods and schemes. Unless specified otherwise, all starting materials used are commercially available.

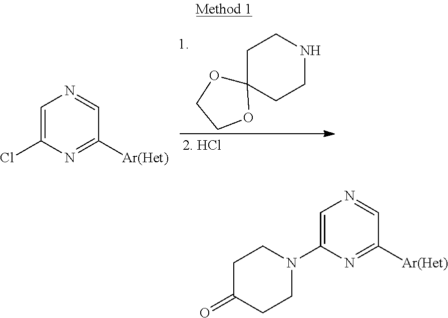

Method 1 is a two-step protocol for the synthesis of 1-(6-(heteroaryl-1-yl)pyrazin-2-yl)piperidin-4-ones or 1-(6-(aryl-1-yl)pyrazin-2-yl)piperidin-4-ones from 2-chloro-6-(heteroaryl-1-yl)pyrazines or 2-chloro-6-(aryl-1-yl)pyrazines. Although the scheme above depicts the synthesis of a substituted pyrazine, the methodology may also be applied to the synthesis of compounds containing heterocycles other than pyrazines. This includes but is not limited to pyrimidines, pyridines, and pyridazines.

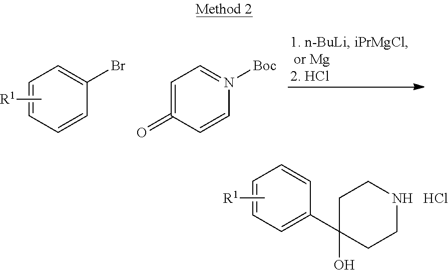

Method 2 is a two-step protocol for the synthesis of 4-(aryl)piperidin-4-ols from aryllithiums or arylmagnesium halides, obtained from metalation of the corresponding arylbromides followed by reaction with a 1-boc-4-piperidinone.

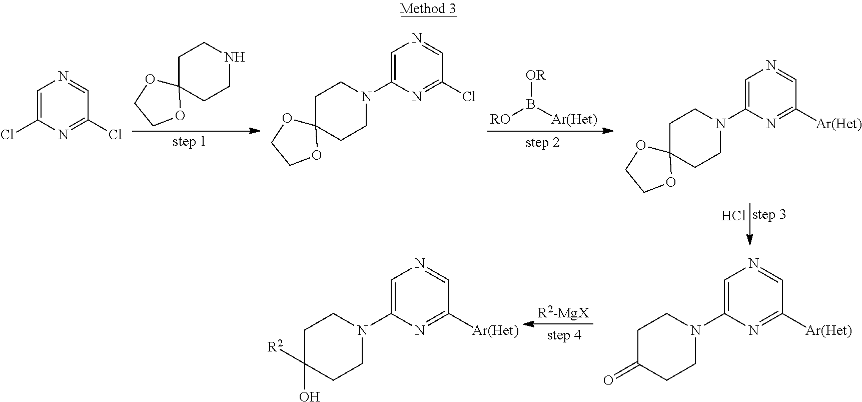

Method 3 is a four-step protocol for the synthesis of 1-(6-(heteroaryl-1-yl)pyrazin-2-yl)-piperidin-4-ols or 1-(6-(aryl-1-yl)pyrazin-2-yl)-piperidin-4-ols from 2, 6-dichloropyrazine. Although the scheme above depicts the synthesis of a substituted pyrazine, the methodology may also be applied to the synthesis of compounds containing heterocycles other than pyrazines. This includes but is not limited to pyrimidines, pyridines, and pyridazines.

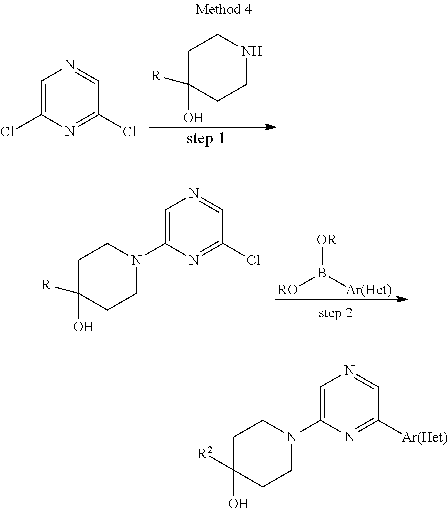

Method 4 is two-step protocol for the synthesis of 1-(6-(heteroaryl-1-yl)pyrazin-2-yl)-piperidin-4-ols or 1-(6-(aryl-1-yl)pyrazin-2-yl)-piperidin-4-ols from 2, 6-dichlorpyrazine. Although the scheme above depicts the synthesis of a substituted pyrazine, the methodology may also be applied to the synthesis of compounds containing heterocycles other than pyrazines. This includes but is not limited to pyrimidines, pyridines, and pyridazines.

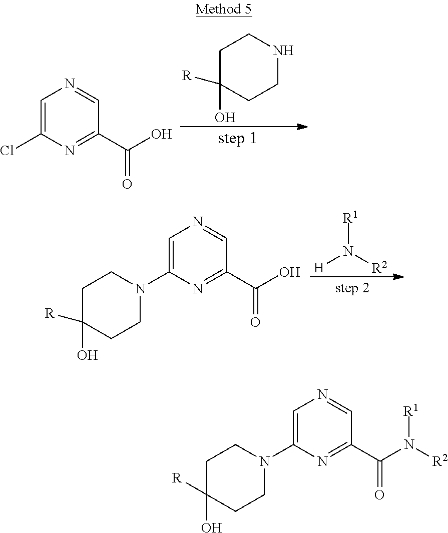

Method 5 is a two-step protocol for the synthesis of 6-(4-substituted-4-hydroxypiperidin-1-yl)-pyrazine-2-carboxamides from 6-chloropyrazine-2-carboxylic acid. Although the scheme above depicts the synthesis of a substituted pyrazine, the methodology may also be applied to the synthesis of compounds containing heterocycles other than pyrazines. This includes but is not limited to pyrimidines, pyridines, and pyridazines.

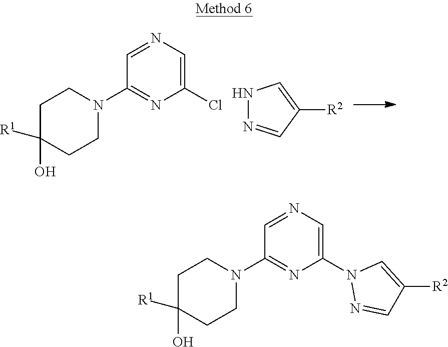

Method 6 is a protocol for the synthesis of 4-substituted-1-(6-(4-substituted-1H-pyrazol-1-yl)pyrazin-2-yl)piperidin-4-ols from 1-(6-chloropyrazin-2-yl)-4-substituted)piperidin-4-ols. Although the scheme above depicts the synthesis of a substituted pyrazine, the methodology may also be applied to the synthesis of compounds containing heterocycles other than pyrazines. This includes but is not limited to pyrimidines, pyridines, and pyridazines.

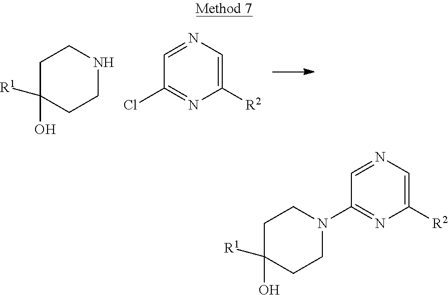

Method 7 is a protocol for the synthesis of substituted pyrazinylpiperidinols. Although the scheme above depicts the synthesis of a substituted pyrazine, the methodology may also be applied to the synthesis of compounds containing heterocycles other than pyrazines. This includes but is not limited to pyrimidines, pyridines, and pyridazines.

The representative examples that follow are intended to help illustrate the present disclosure, and are not intended to, nor should they be construed to, limit the scope of the invention.

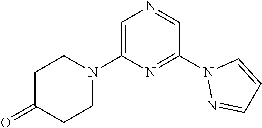 [SMILES: O=C1CCN(c2cncc(-n3cccn3)n2)CC1]

### Method 1. 1-(6-(1H-pyrazol-1-yl)pyrazin-2-yl)piperidin-4-one

1,4-Dioxa-8-azaspiro[4.5]decane (1.56 g, 10.9 mmol), 2-chloro-6-(1H-pyrazol-1-yl)pyrazine (1.80 g, 9.96 mmol), and potassium carbonate (2.75 g, 19.9 mmol) were combined in DMF (10 mL) and heated to 90° C. for 1 h. The reaction was cooled to room temperature and diluted with ethyl acetate and brine. The organic layer was washed with brine 3 more times. The organic extracts were dried over Na₂SO₄, filtered, and concentrated. This material was then dissolved in 20 mL of acetone and treated with 20 mL of 1N HCl. The reaction mixture was heated to 50° C. overnight. The organic solvent was removed under reduced pressure and the pH was adjusted to 12 with 6N NaOH solution. The product was extracted with DCM. The residue was taken up in DCM and the organic layer was washed with saturated NaHCO₃ solution. The organic layer was then dried over Na₂SO₄, filtered and concentrated. The residue was purified by silica gel chromatography (Biotage 80 g silica cartridge; 0-75% ethyl acetate in heptanes) to afford the title compound (1.45 g, 60%). LCMS: m/z=244.1 [M+1].

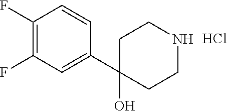 [SMILES: OC1(c2ccc(F)c(F)c2)CCNCC1 ; Cl]

### Method 2. 4-(3,4-difluorophenyl)piperidin-4-ol.HCl

A solution of 4-bromo-1,2-difluorobenzene (2.37 mL, 21 mmol) in THF (50 mL) cooled to −78° C. was treated with n-butyllithium (13.1 mL, 21.0 mmol). The mixture was stirred at −78° C. for 1 h and then tert-butyl 4-oxopiperidine-1-carboxylate (3.98 g, 20 mmol) was added as a solution in THF (10 mL). The mixture was stirred at −78° C. for 1 h and then warmed to 0° C. before being quenched with saturated aqueous NH₄Cl solution. The product was extracted with ethyl acetate. The organic extract was washed with brine, dried over Na₂SO₄, filtered, and concentrated. The residue was purified by silica gel chromatography (Biotage 120 g silica cartridge; 0-35% EA in heptanes) to obtain the intermediate, tert-butyl 4-(3,4-difluorophenyl)-4-hydroxypiperidine-1-carboxylate, as a thick oil (3.05 g, 49%). LCMS: m/z=336.1 [M+Na].

tert-butyl 4-(3,4-difluorophenyl)-4-hydroxypiperidine-1-carboxylate (3.05 g, 9.73 mmol) was dissolved in HCl (10 mL, 40.0 mmol; 4M in dioxane) and the mixture was stirred until the reaction was complete as judged by LCMS. The mixture was concentrated under reduced pressure to obtain the title compound as a yellow solid (2.35 g, 97%). LCMS: m/z=214.1 [M+1].

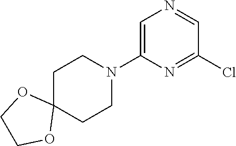 [SMILES: Clc1cncc(N2CCC3(CC2)OCCO3)n1]

### Method 3, Step 1. 8-(6-chloropyrazin-2-yl)-1,4-dioxa-8-azaspiro[4.5]decane

1,4-dioxa-8-azaspiro[4.5]decane (3.15 g, 22.0 mmol), 2,6-dichloropyrazine (2.97 g, 20 mmol), and potassium carbonate (5.52 g, 40.0 mmol) were combined in DMF (20 mL) and heated to 55° C. for 16 h. The reaction was cooled to room temperature and diluted with ethyl acetate and brine. The organic layer was washed with brine three more times. The organic extracts were dried over Na₂SO₄, filtered, and concentrated. The residue was purified by silica gel chromatography (biotage 80 g silica cartridge; 0-60% EA in heptanes) to afford the title compound (4.51 g, 88%). LCMS: m/z=256.1 [M+1].

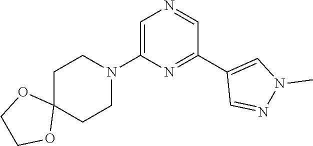 [SMILES: Cn1cc(-c2cncc(N3CCC4(CC3)OCCO4)n2)cn1]

### Method 3, Step 2. 8-(6-(1-methyl-1H-pyrazol-4-yl)pyrazin-2-yl)-1,4-dioxa-8-azaspiro[4.5]decane

8-(6-chloropyrazin-2-yl)-1,4-dioxa-8-azaspiro[4.5]decane (1 g, 3.91 mmol), 1-methyl-4-(4,4,5,5-tetramethyl-1,3,2-dioxaborolan-2-yl)-1H-pyrazole (2.43 g, 11.7 mmol), S-phos 3rd generation precatalyst (91.2 mg, 117 μmol), potassium phosphate (2.48 g, 11.7 mmol), were combined in degassed dioxane (10 mL) and water (2 mL) and the mixture was heated to 200° C. under an atmosphere of nitrogen for 5 minutes. The reaction was diluted with ethyl acetate and filtered through a pad of celite eluting with ethyl acetate. The eluent was concentrated and the residue was purified by silica gel chromatography (0-100% 3:1 ethyl acetate:EtOH in heptanes) to afford the title compound (1.09 g, 93%). LCMS: m/z=302.2 [M+1].

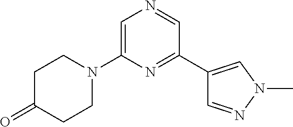 [SMILES: Cn1cc(-c2cncc(N3CCC(=O)CC3)n2)cn1]

### Method 3, Step 3. 1-(6-(1-methyl-1H-pyrazol-4-yl)pyrazin-2-yl)piperidin-4-one

A solution of 8-[6-(1-methyl-1H-pyrazol-4-yl)pyrazin-2-yl]-1,4-dioxa-8-azaspiro[4.5]decane (1.09 g, 3.61 mmol) in 6N HCl (3.00 mL, 18.0 mmol) and acetone (10 mL) was heated to 55° C. overnight. The reaction was cooled to room temperature and the acetone was removed under reduced pressure. The pH of the solution was adjusted to approximately 12 with aqueous 6N NaOH solution and the product was extracted with DCM. The organic extracts were dried over Na₂SO₄, filtered, and concentrated. The residue was purified by silica gel chromatography (Biotage 30 g silica cartridge; 0-100% 3:1 EA:EtOH in heptanes) to afford the title compound (780 mg, 84%). LCMS: m/z=258.2 [M+1].

### Example 1

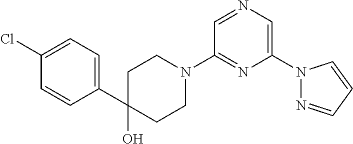 [SMILES: OC1(c2ccc(Cl)cc2)CCN(c2cncc(-n3cccn3)n2)CC1]

### 1-(6-(1H-pyrazol-1-yl)pyrazin-2-yl)-4-(4-chlorophenyl)piperidin-4-ol

### Method 3, Step 4. 1-(6-(1H-pyrazol-1-yl)pyrazin-2-yl)-4-(4-chlorophenyl)piperidin-4-ol

Bromo(4-chlorophenyl)magnesium (410 μL, 410 μmol) was added to a 0° C. solution of 1-[6-(1H-pyrazol-1-yl)pyrazin-2-yl]piperidin-4-one (50 mg, 205 μmol) in THF (1 mL). The reaction was quenched with saturated aqueous NH₄Cl solution after 15 minutes. The product was extracted with ethyl acetate. The organic extracts were dried over Na₂SO₄, filtered, and concentrated. The residue was purified by silica gel chromatography (Biotage 10 g silica cartridge; 0-50% 3:1 ethyl acetate:EtOH in heptane) to afford the title compound (39 mg, 53%). ¹H NMR (400 MHz, DMSO-d6) δ=8.59 (d, J=2.9 Hz, 1H), 8.29 (m, 2H), 7.80 (d, J=1.5 Hz, 1H), 7.58-7.45 (m, 2H), 7.40-7.28 (m, 2H), 6.59-6.50 (m, 1H), 4.36 (m, 2H), 3.42-3.31 (m, 2H), 2.02-1.88 (m, 2H), 1.69 (m, 2H). LCMS: m/z=356.1 [M+1].

The compounds in Table 1 were prepared using similar procedures to those described for Example 1.

TABLE 1

| | | | Exact |
|---|---|---|---|
| Ex | | | Mass |
| Nos. | Structure | IUPAC Name | [M + 1] |
| 2 | 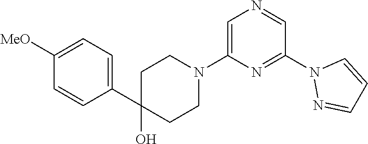 [SMILES: COc1ccc(C2(O)CCN(c3cncc(-n4cccn4)n3)CC2)cc1] | 1-(6-(1H-pyrazol-1- yl)pyrazin-2-yl)-4-(4- methoxyphenyl) piperidin-4-ol | Calc'd 352.2, Found 352.2 |
| 3 | 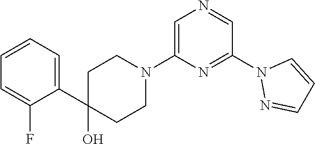 [SMILES: OC1(c2ccccc2F)CCN(c2cncc(-n3cccn3)n2)CC1] | 1-(6-(1H-pyrazol-1- yl)pyrazin-2-yl)-4-(2- fluorophenyl)piperidin- 4-ol | Calc'd 340.2, Found 340.2 |
| 4 | 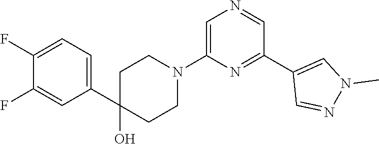 [SMILES: Cn1cc(-c2cncc(N3CCC(O)(c4ccc(F)c(F)c4)CC3)n2)cn1] | 4-(3,4-difluorophenyl)- 1-(6-(1-methyl-1H- pyrazol-4-yl)pyrazin-2- yl)piperidin-4-ol | Calc'd 372.2, Found 372.1 |
| 5 | 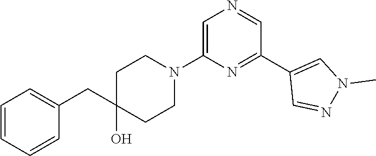 [SMILES: Cn1cc(-c2cncc(N3CCC(O)(Cc4ccccc4)CC3)n2)cn1] | 4-benzyl-1-(6-(1- methyl-1H-pyrazol-4- yl)pyrazin-2- yl)piperidin-4-ol | Calc'd 350.2, Found 350.2 |
| 6 | 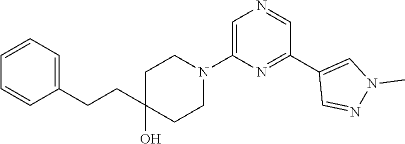 [SMILES: Cn1cc(-c2cncc(N3CCC(O)(CCc4ccccc4)CC3)n2)cn1] | 1-(6-(1-methyl-1H- pyrazol-4-yl)pyrazin-2- yl)-4- phenethylpiperidin-4-ol | Calc'd 364.2, Found 364.2 |

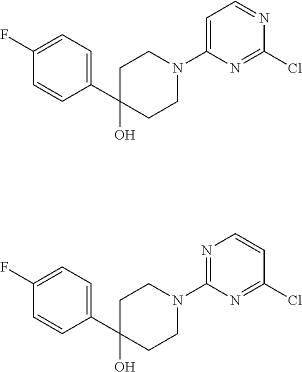 [SMILES: OC1(c2ccc(F)cc2)CCN(c2ccnc(Cl)n2)CC1 ; OC1(c2ccc(F)cc2)CCN(c2nccc(Cl)n2)CC1]

### Method 4, Step 1. 1-(2-chloropyrimidin-4-yl)-4-(4-fluorophenyl)piperidin-4-ol and 1-(4-chloropyrimidin-2-yl)-4-(4-fluorophenyl)piperidin-4-ol

4-(4-fluorophenyl)piperidin-4-ol (431 mg, 2.21 mmol), 2,4-dichloropyrimidine (300 mg, 2.01 mmol), and potassium carbonate (555 mg, 4.02 mmol) were combined in acetonitrile (4 ML) and heated to 50° C. for 16 h. The reaction was filtered through celite eluting with ethyl acetate and the eluent was concentrated. The reaction was cooled to room temperature and directly purified by reverse phase chromatography (Biotage 30 g C18 cartridge; 5-90% ACN in water+0.1% TFA). The fractions containing the two products were concentrated. The residues from both fractions were partitioned between DCM and saturated NaHCO₃ solution. The organic layers were dried over Na₂SO₄, filtered, and concentrated. The fractions containing faster-eluting isomer, 1-(2-chloropyrimidin-4-yl)-4-(4-fluorophenyl)piperidin-4-ol, were impure after column chromatography. Pure 1-(2-chloropyrimidin-4-yl)-4-(4-fluorophenyl)piperidin-4-ol (275 mg, 44%) was obtained by recrystallization from hot DCM. LCMS: m/z=308.1 [M+1]. The slower-eluting isomer 1-(4-chloropyrimidin-2-yl)-4-(4-fluorophenyl)piperidin-4-ol (54 mg, 9%) was pure after reverse phase purification. LCMS: m/z=308.1 [M+1].

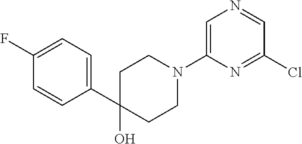 [SMILES: OC1(c2ccc(F)cc2)CCN(c2cncc(Cl)n2)CC1]

### Method 4, Step 1. 1-(6-chloropyrazin-2-yl)-4-(fluorophenyl)piperidin-4-ol

4-(4-fluorophenyl)piperidin-4-ol (212 mg, 1.09 mmol), 2,6-dichloropyrazine (148 mg, 993 μmol), and potassium carbonate (273 mg, 1.98 mmol) were combined in acetonitrile (2 mL) and heated to 40° C. for 24 h. The reaction was cooled to room temperature and directly purified by reverse phase chromatography (Biotage 30 g C18 cartridge; 5-90% ACN in water+0.1% TFA). The fractions containing product were concentrated. The residue was taken up in DCM and the organic layer was washed with saturated NaHCO₃ solution. The organic layer was then dried over Na₂SO₄, filtered and concentrated to afford the title compound (150 mg, 49%). LCMS: m/z=308.1 [M+1].

### Example 7

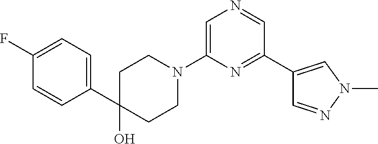 [SMILES: Cn1cc(-c2cncc(N3CCC(O)(c4ccc(F)cc4)CC3)n2)cn1]

### 4-(4-fluorophenyl)-1-(6-(1-methyl-1H-pyrazol-4-yl)pyrazin-2-yl)piperidin-4-ol

### Method 4, Step 2. 4-(4-fluorophenyl)-1-(6-(1-methyl-1H-pyrazol-4-yl)pyrazin-2-yl)piperidin-4-ol

Degassed dioxane (0.4 mL) and water (60 μL) were added to a mixture of 1-(6-chloropyrazin-2-yl)-4-(4-fluorophenyl)piperidin-4-ol (50 mg, 162 μmol), 1-methyl-4-(4,4,5,5-tetramethyl-1,3,2-dioxaborolan-2-yl)-1H-pyrazole (100 mg, 485 μmol), S-phos 3rd generation precatalyst (12.6 mg, 16.2 μmol), and cesium carbonate (157 mg, 485 μmol) in a resealable screw top test tube. The tube was sealed and the mixture was stirred overnight at 80° C. under an atmosphere of nitrogen. The reaction mixture was cooled to room temperature, diluted with ethyl acetate, and filtered through a pad of celite. The eluent was concentrated and the residue was purified by silica gel chromatography (Biotage 10 g silica cartridge; 0-75% 3:1 ethyl acetate:EtOH in heptane) to afford the title compound (36 mg, 63%). ¹H NMR (400 MHz, DMSO-d6) δ=8.26 (s, 1H), 8.10 (s, 2H), 7.97 (s, 1H), 7.60-7.41 (m, 2H), 7.18-7.03 (m, 2H), 5.19 (s, 1H), 4.31 (m, 2H), 3.86 (m, 3H), 3.30-3.25 (m, 2H), 2.00-1.86 (m, 2H), 1.69 (in, 2H). LCMS: m/z=354.2 [M+1].

The compounds in Table 2 were prepared using similar procedures to those described for Example 7 using the appropriate starting materials.

TABLE 2

| | | | Exact |
|---|---|---|---|
| Ex | | | Mass |
| Nos. | Structure | IUPAC Name | [M + 1] |
| 8 | 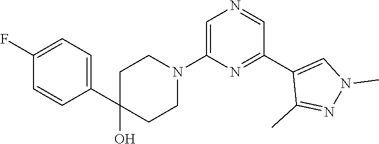 [SMILES: Cc1nn(C)cc1-c1cncc(N2CCC(O)(c3ccc(F)cc3)CC2)n1] | 1-(6-(1,3-dimethyl-1H- pyrazol-4-yl)pyrazin-2- yl)-4-(4- fluorophenyl)piperidin- 4-ol | Calc'd 368.2, Found 368.2 |
| 9 | 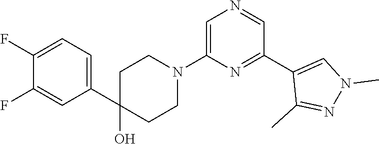 [SMILES: Cc1nn(C)cc1-c1cncc(N2CCC(O)(c3ccc(F)c(F)c3)CC2)n1] | 4-(3,4-difluorophenyl)- 1-(6-(1,3-dimethyl-1H- pyrazol-4-yl)pyrazin-2- yl)piperidin-4-ol | Calc'd 386.2, Found 386.2 |
| 10 | 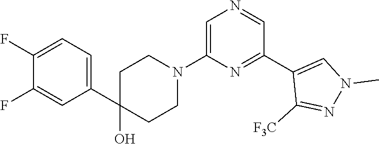 [SMILES: Cn1cc(-c2cncc(N3CCC(O)(c4ccc(F)c(F)c4)CC3)n2)c(C(F)(F)F)n1] | 4-(3,4-difluorophenyl)- 1-(6-(1-methyl-3- (trifluoromethyl)-1H- pyrazol-4-yl)pyrazin-2- yl)piperidin-4-ol | Calc'd 440.1, Found 440.1 |
| 11 | 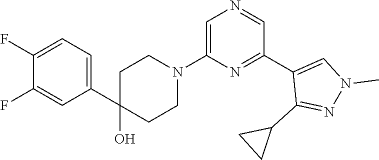 [SMILES: Cn1cc(-c2cncc(N3CCC(O)(c4ccc(F)c(F)c4)CC3)n2)c(C2CC2)n1] | 1-(6-(3-cyclopropyl-1- methyl-1H-pyrazol-4- yl)pyrazin-2-yl)-4-(3,4- difluorophenyl)piperidin- 4-ol | Calc'd 412.2, Found 412.2 |
| 12 | 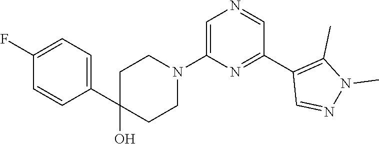 [SMILES: Cc1c(-c2cncc(N3CCC(O)(c4ccc(F)cc4)CC3)n2)cnn1C] | 1-(6-(1,5-dimethyl-1H- pyrazol-4-yl)pyrazin-2- yl)-4-(4- fluorophenyl)piperidin- 4-ol | Calc'd 368.2, Found 368.2 |
| 13 | 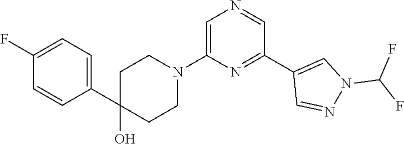 [SMILES: OC1(c2ccc(F)cc2)CCN(c2cncc(-c3cnn(C(F)F)c3)n2)CC1] | 1-(6-(1- (difluoromethyl)-1H- pyrazol-4-yl)pyrazin-2- yl)-4-(4- fluorophenyl)piperidin- 4-ol | Calc'd 390.2, Found 390.2 |
| 14 | 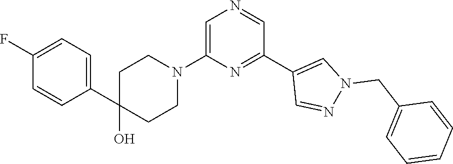 [SMILES: OC1(c2ccc(F)cc2)CCN(c2cncc(-c3cnn(Cc4ccccc4)c3)n2)CC1] | 1-(6-(1-benzyl-1H- pyrazol-4-yl)pyrazin-2- yl)-4-(4- fluorophenyl)piperidin- 4-ol | Calc'd 430.2, Found 430.2 |
| 15 | 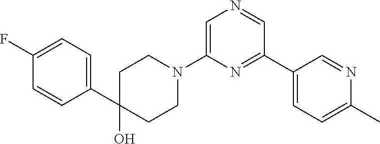 [SMILES: Cc1ccc(-c2cncc(N3CCC(O)(c4ccc(F)cc4)CC3)n2)cn1] | 4-(4-fluorophenyl)-1-(6- (6-methylpyridin-3- yl)pyrazin-2- yl)piperidin-4-ol | Calc'd 365.2, Found 365.2 |
| 16 | 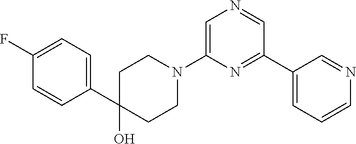 [SMILES: OC1(c2ccc(F)cc2)CCN(c2cncc(-c3cccnc3)n2)CC1] | 4-(4-fluorophenyl)-1-(6- (pyridin-3-yl)pyrazin-2- yl)piperidin-4-ol | Calc'd 351.2, Found 351.2 |
| 17 | 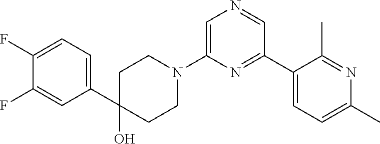 [SMILES: Cc1ccc(-c2cncc(N3CCC(O)(c4ccc(F)c(F)c4)CC3)n2)c(C)n1] | 4-(3,4-difluorophenyl)- 1-(6-(2,6- dimethylpyridin-3- yl)pyrazin-2- yl)piperidin-4-ol | Calc'd 397.2, Found 397.2 |
| 18 | 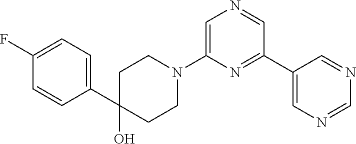 [SMILES: OC1(c2ccc(F)cc2)CCN(c2cncc(-c3cncnc3)n2)CC1] | 4-(4-fluorophenyl)-1-(6- (pyrimidin-5-yl)pyrazin- 2-yl)piperidin-4-ol | Calc'd 352.2, Found 352.2 |
| 19 | 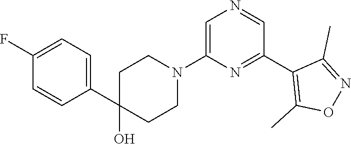 [SMILES: Cc1noc(C)c1-c1cncc(N2CCC(O)(c3ccc(F)cc3)CC2)n1] | 1-(6-(3,5- dimethylisoxazol-4- yl)pyrazin-2-yl)-4-(4- fluorophenyl)piperidin- 4-ol | Calc'd 369.2, Found 369.2 |
| 20 | 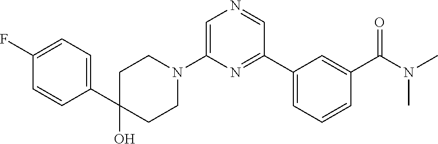 [SMILES: CN(C)C(=O)c1cccc(-c2cncc(N3CCC(O)(c4ccc(F)cc4)CC3)n2)c1] | 3-(6-(4-(4- fluorophenyl)-4- hydroxypiperidin-1- yl)pyrazin-2-yl)-N,N- dimethylbenzamide | Calc'd 421.2, Found 421.2 |
| 21 | 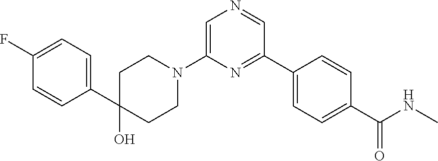 [SMILES: CNC(=O)c1ccc(-c2cncc(N3CCC(O)(c4ccc(F)cc4)CC3)n2)cc1] | 4-(6-(4-(4- fluorophenyl)-4- hydroxypiperidin-1- yl)pyrazin-2-yl)-N- methylbenzamide | Calc'd 407.2, Found 407.2 |
| 22 | 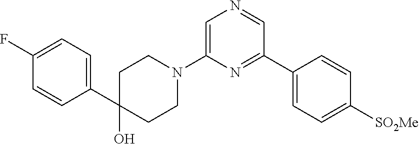 [SMILES: CS(=O)(=O)c1ccc(-c2cncc(N3CCC(O)(c4ccc(F)cc4)CC3)n2)cc1] | 4-(4-fluorophenyl)-1-(6- (4- (methylsulfonyl)phenyl) pyrazin-2-yl)piperidin-4- ol | Calc'd 428.2, Found 428.2 |
| 23 | 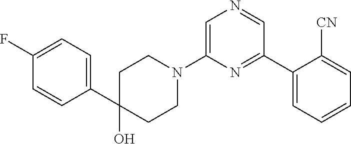 [SMILES: N#Cc1ccccc1-c1cncc(N2CCC(O)(c3ccc(F)cc3)CC2)n1] | 2-(6-(4-(4- fluorophenyl)-4- hydroxypiperidin-1- yl)pyrazin-2- yl)benzonitrile | Calc'd 375.2, Found 375.2 |
| 24 | 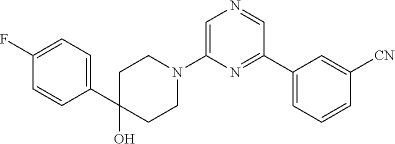 [SMILES: N#Cc1cccc(-c2cncc(N3CCC(O)(c4ccc(F)cc4)CC3)n2)c1] | 3-(6-(4-(4- fluorophenyl)-4- hydroxypiperidin-1- yl)pyrazin-2- yl)benzonitrile | Calc'd 375.2, Found 375.2 |
| 25 | 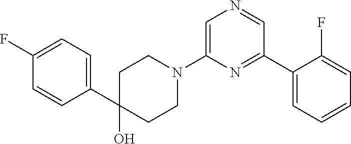 [SMILES: OC1(c2ccc(F)cc2)CCN(c2cncc(-c3ccccc3F)n2)CC1] | 4-(4-fluorophenyl)-1-(6- (2-fluorophenyl)pyrazin- 2-yl)piperidin-4-ol | Calc'd 368.2, Found 368.2 |
| 26 | 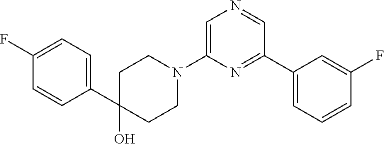 [SMILES: OC1(c2ccc(F)cc2)CCN(c2cncc(-c3cccc(F)c3)n2)CC1] | 4-(4-fluorophenyl)-1-(6- (3-fluorophenyl)pyrazin- 2-yl)piperidin-4-ol | Calc'd 368.2, Found 368.2 |
| 27 | 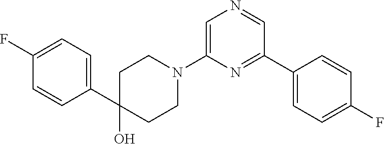 [SMILES: OC1(c2ccc(F)cc2)CCN(c2cncc(-c3ccc(F)cc3)n2)CC1] | 4-(4-fluorophenyl)-1-(6- (4-fluorophenyl)pyrazin- 2-yl)piperidin-4-ol | Calc'd 368.2, Found 368.2 |
| 28 | 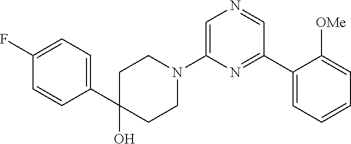 [SMILES: COc1ccccc1-c1cncc(N2CCC(O)(c3ccc(F)cc3)CC2)n1] | 4-(4-fluorophenyl)-1-(6- (2- methoxyphenyl)pyrazin- 2-yl)piperidin-4-ol | Calc'd 380.2, Found 380.2 |
| 29 | 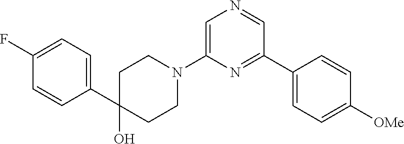 [SMILES: COc1ccc(-c2cncc(N3CCC(O)(c4ccc(F)cc4)CC3)n2)cc1] | 4-(4-fluorophenyl)-1-(6- (4- methoxyphenyl)pyrazin- 2-yl)piperidin-4-ol | Calc'd 380.2, Found 380.2 |
| 30 | 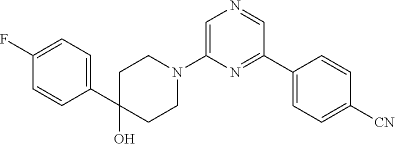 [SMILES: N#Cc1ccc(-c2cncc(N3CCC(O)(c4ccc(F)cc4)CC3)n2)cc1] | 4-(6-(4-(4- fluorophenyl)-4- hydroxypiperidin-1- yl)pyrazin-2- yl)benzonitrile | Calc'd 375.2, Found 375.2 |
| 31 | 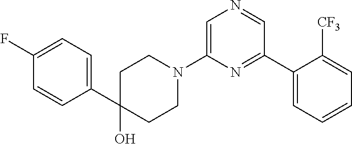 [SMILES: OC1(c2ccc(F)cc2)CCN(c2cncc(-c3ccccc3C(F)(F)F)n2)CC1] | 4-(4-fluorophenyl)-1-(6- (2- (trifluoromethyl)phenyl) pyrazin-2-yl)piperidin-4- ol | Calc'd 418.2, Found 418.2 |
| 32 | 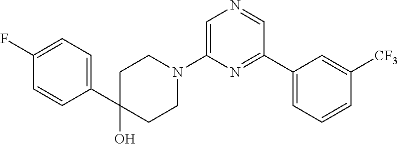 [SMILES: OC1(c2ccc(F)cc2)CCN(c2cncc(-c3cccc(C(F)(F)F)c3)n2)CC1] | 4-(4-fluorophenyl)-1-(6- (3- (trifluoromethyl)phenyl) pyrazin-2-yl)piperidin-4- ol | Calc'd 418.2, Found 418.1 |
| 33 | 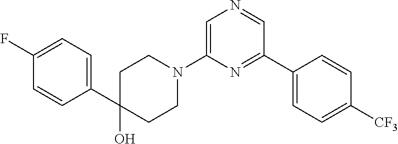 [SMILES: OC1(c2ccc(F)cc2)CCN(c2cncc(-c3ccc(C(F)(F)F)cc3)n2)CC1] | 4-(4-fluorophenyl)-1-(6- (4- (trifluoromethyl)phenyl) pyrazin-2-yl)piperidin- 4-ol | Calc'd 418.2, Found 418.2 |
| 34 | 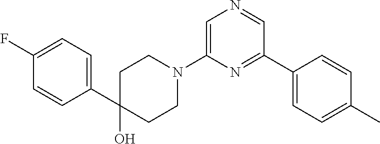 [SMILES: Cc1ccc(-c2cncc(N3CCC(O)(c4ccc(F)cc4)CC3)n2)cc1] | 4-(4-fluorophenyl)-1-(6- (p-tolyl)pyrazin-2- yl)piperidin-4-ol | Calc'd 364.2, Found 364.2 |
| 35 | 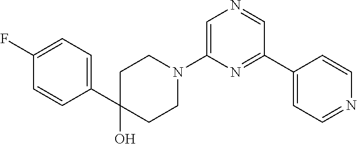 [SMILES: OC1(c2ccc(F)cc2)CCN(c2cncc(-c3ccncc3)n2)CC1] | 4-(4-fluorophenyl)-1-(6- (pyridin-4-yl)pyrazin-2- yl)piperidin-4-ol | Calc'd 351.2, Found 351.1 |
| 36 | 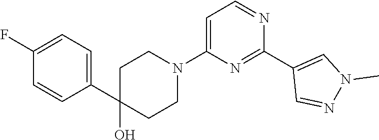 [SMILES: Cn1cc(-c2nccc(N3CCC(O)(c4ccc(F)cc4)CC3)n2)cn1] | 4-(4-fluorophenyl)-1-(2- (1-methyl-1H-pyrazol-4- yl)pyrimidin-4- yl)piperidin-4-ol | Calc'd 354.2, Found 354.2 |
| 37 | 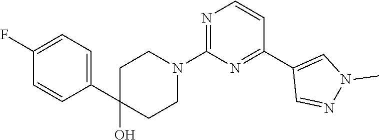 [SMILES: Cn1cc(-c2ccnc(N3CCC(O)(c4ccc(F)cc4)CC3)n2)cn1] | 4-(4-fluorophenyl)-1-(4- (1-methyl-1H-pyrazol-4- yl)pyrimidin-2- yl)piperidin-4-ol | Calc'd 354.2, Found 354.2 |
| 38 | 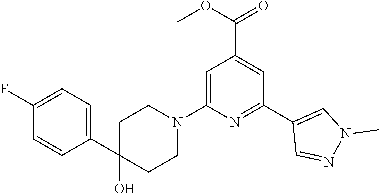 [SMILES: COC(=O)c1cc(-c2cnn(C)c2)nc(N2CCC(O)(c3ccc(F)cc3)CC2)c1] | methyl 2-(4-(4- fluorophenyl)-4- hydroxypiperidin-1-yl)- 6-(1-methyl-1H-pyrazol- 4-yl)isonicotinate | Calc'd 411.2, Found 411.2 |
| 39 | 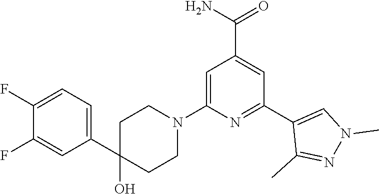 [SMILES: Cc1nn(C)cc1-c1cc(C(N)=O)cc(N2CCC(O)(c3ccc(F)c(F)c3)CC2)n1] | 2-(4-(3,4- difluorophenyl)-4- hydroxypiperidin-1-yl)- 6-(1,3-dimethyl-1H- pyrazol-4- yl)isonicotinamide | Calc'd 428.2, Found 428.7 |
| 40 | 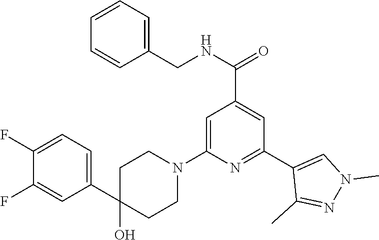 [SMILES: Cc1nn(C)cc1-c1cc(C(=O)NCc2ccccc2)cc(N2CCC(O)(c3ccc(F)c(F)c3)CC2)n1] | N-benzyl-2-(4-(3,4- difluorophenyl)-4- hydroxypiperidin-1-yl)- 6-(1,3-dimethyl-1H- pyrazol-4- yl)isonicotinamide | Calc'd 518.2, Found 518.4 |
| 41 | 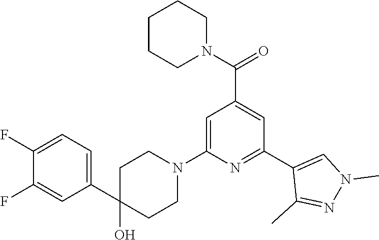 [SMILES: Cc1nn(C)cc1-c1cc(C(=O)N2CCCCC2)cc(N2CCC(O)(c3ccc(F)c(F)c3)CC2)n1] | (2-(4-(3,4- difluorophenyl)-4- hydroxypiperidin-1-yl)- 6-(1,3-dimethyl-1H- pyrazol-4-yl)pyridin-4- yl)(piperidin-1- yl)methanone | Calc'd 496.2, Found 496.4 |
| 42 | 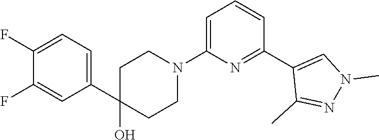 [SMILES: Cc1nn(C)cc1-c1cccc(N2CCC(O)(c3ccc(F)c(F)c3)CC2)n1] | 4-(3,4-difluorophenyl)- 1-(6-(1,3-dimethyl-1H- pyrazol-4-yl)pyridin-2- yl)piperidin-4-ol | Calc'd 385.2, Found 385.2 |
| 43 | 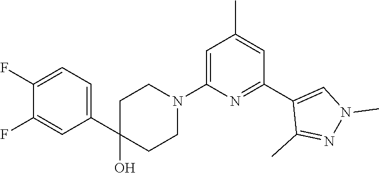 [SMILES: Cc1cc(-c2cn(C)nc2C)nc(N2CCC(O)(c3ccc(F)c(F)c3)CC2)c1] | 4-(3,4-difluorophenyl)- 1-(6-(1,3-dimethyl-1H- pyrazol-4-yl)-4- methylpiperidin-2- yl)piperidin-4-ol | Calc'd 399.2, Found 399.5 |
| 44 | 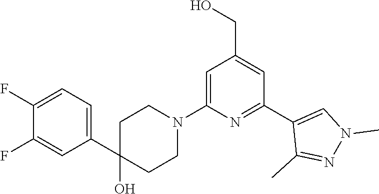 [SMILES: Cc1nn(C)cc1-c1cc(CO)cc(N2CCC(O)(c3ccc(F)c(F)c3)CC2)n1] | 4-(3,4-difluorophenyl)- 1-(6-(1,3-dimethyl-1H- pyrazol-4-yl)-4- (hydroxymethyl)pyridin- 2-yl)piperidin-4-ol | Calc'd 415.2, Found 415.3 |
| 45 | 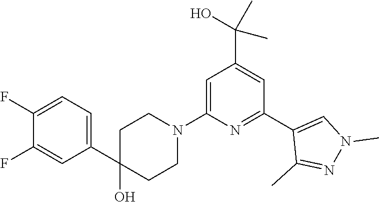 [SMILES: Cc1nn(C)cc1-c1cc(C(C)(C)O)cc(N2CCC(O)(c3ccc(F)c(F)c3)CC2)n1] | 4-(3,4-difluorophenyl)- 1-(6-(1,3-dimethyl-1H- pyrazol-4-yl)-4-(2- hydroxypropan-2- yl)pyridin-2- yl)piperidin-4-ol | Calc'd 443.2, Found 443.7 |
| 46 | 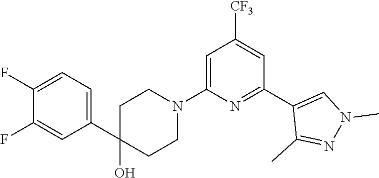 [SMILES: Cc1nn(C)cc1-c1cc(C(F)(F)F)cc(N2CCC(O)(c3ccc(F)c(F)c3)CC2)n1] | 4-(3,4-difluorophenyl)- 1-(6-(1,3-dimethyl-1H- pyrazol-4-yl)-4- (trifluoromethyl)pyridin- 2-yl)piperidin-4-ol | Calc'd 453.2, Found 453.3 |
| 47 | 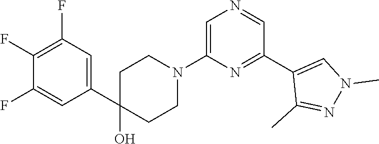 [SMILES: Cc1nn(C)cc1-c1cncc(N2CCC(O)(c3cc(F)c(F)c(F)c3)CC2)n1] | 1-(6-(1,3-dimethyl-1H- pyrazol-4-yl)pyrazin-2- yl)-4-(3,4,5- trifluorophenyl)piperidin- 4-ol | Calc'd 404.2, Found 404.1 |
| 48 | 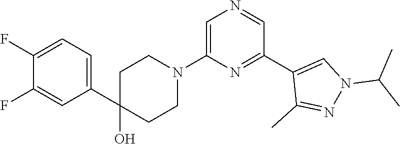 [SMILES: Cc1nn(C(C)C)cc1-c1cncc(N2CCC(O)(c3ccc(F)c(F)c3)CC2)n1] | 4-(3,4-difluorophenyl)- 1-(6-(1-isopropyl-3- methyl-1H-pyrazol-4- yl)pyrazin-2- yl)piperidin-4-ol | Calc'd 414.2, Found 414.2 |
| 49 | 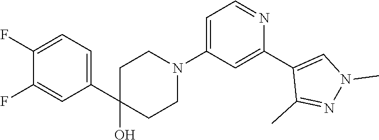 [SMILES: Cc1nn(C)cc1-c1cc(N2CCC(O)(c3ccc(F)c(F)c3)CC2)ccn1] | 4-(3,4-difluorophenyl)- 1-(2-(1,3-dimethyl-1H- pyrazol-4-yl)pyridin-4- yl)piperidin-4-ol | Calc'd 385.2, Found 385.2 |
| 50 | 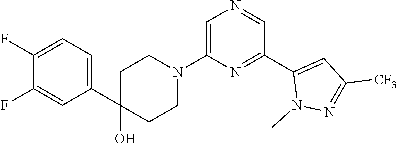 [SMILES: Cn1nc(C(F)(F)F)cc1-c1cncc(N2CCC(O)(c3ccc(F)c(F)c3)CC2)n1] | 4-(3,4-difluorophenyl)- 1-(6-(1-methyl-3- (trifluoromethyl)-1H- pyrazol-5-yl)pyrazin-2- yl)piperidin-4-ol | Calc'd 440.1, Found 440.1 |
| 51 | 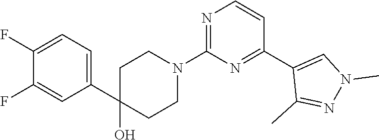 [SMILES: Cc1nn(C)cc1-c1ccnc(N2CCC(O)(c3ccc(F)c(F)c3)CC2)n1] | 4-(3,4-difluorophenyl)- 1-(4-(1,3-dimethyl-1H- pyrazol-4-yl)pyrimidin- 2-yl)piperidin-4-ol | Calc'd 386.2, Found 386.2 |
| 52 | 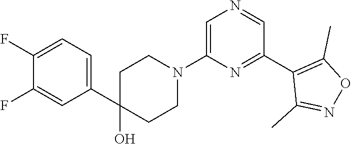 [SMILES: Cc1noc(C)c1-c1cncc(N2CCC(O)(c3ccc(F)c(F)c3)CC2)n1] | 4-(3,4-difluorophenyl)- 1-(6-(3,5- dimethylisoxazol-4- yl)pyrazin-2- yl)piperidin-4-ol | Calc'd 387.2, Found 387.2 |
| 53 | 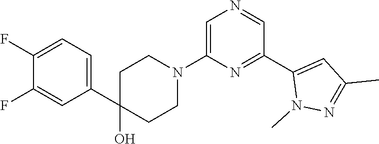 [SMILES: Cc1cc(-c2cncc(N3CCC(O)(c4ccc(F)c(F)c4)CC3)n2)n(C)n1] | 4-(3,4-difluorophenyl)- 1-(6-(1,3-dimethyl-1H- pyrazol-5-yl)pyrazin-2- yl)piperidin-4-ol | Calc'd 386.2, Found 386.2 |
| 54 | 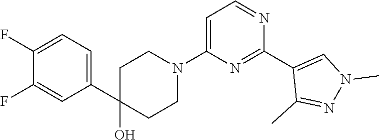 [SMILES: Cc1nn(C)cc1-c1nccc(N2CCC(O)(c3ccc(F)c(F)c3)CC2)n1] | 4-(3,4-difluorophenyl)- 1-(2-(1,3-dimethyl-1H- pyrazol-4-yl)pyrimidin- 4-yl)piperidin-4-ol | Calc'd 386.2, Found 386.2 |
| 55 | 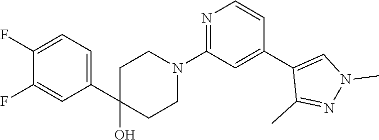 [SMILES: Cc1nn(C)cc1-c1ccnc(N2CCC(O)(c3ccc(F)c(F)c3)CC2)c1] | 4-(3,4-difluorophenyl)- 1-(4-(1,3-dimethyl-1H- pyrazol-4-yl)pyridin-2- yl)piperidin-4-ol | Calc'd 385.2, Found 385.2 |
| 56 | 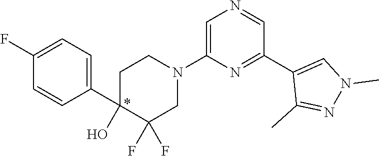 [SMILES: Cc1nn(C)cc1-c1cncc(N2CCC(O)(c3ccc(F)cc3)C(F)(F)C2)n1] | 1-(6-(1,3-dimethyl-1H- pyrazol-4-yl)pyrazin-2- yl)-3,3-difluoro-4-(4- fluorophenyl)piperidin- 4-ol | Calc'd 440.2, Found 404.1 |
| 57 | 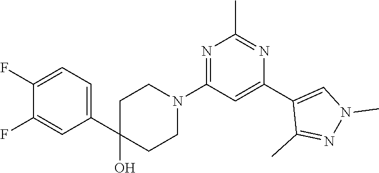 [SMILES: Cc1nc(-c2cn(C)nc2C)cc(N2CCC(O)(c3ccc(F)c(F)c3)CC2)n1] | 4-(3,4-difluorophenyl)- 1-(6-(1,3-dimethyl-1H- pyrazol-4-yl)-2- methylpyrimidin-4- yl)piperidin-4-ol | Calc'd 400.2, Found 400.6 |
| 58 | 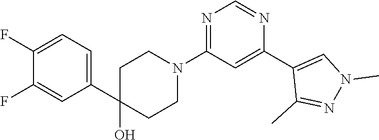 [SMILES: Cc1nn(C)cc1-c1cc(N2CCC(O)(c3ccc(F)c(F)c3)CC2)ncn1] | 4-(3,4-difluorophenyl)- 1-(6-(1,3-dimethyl-1H- pyrazol-4-yl)pyrimidin- 4-yl)piperidin-4-ol | Calc'd 386.2, Found 386.4 |
| 59 | 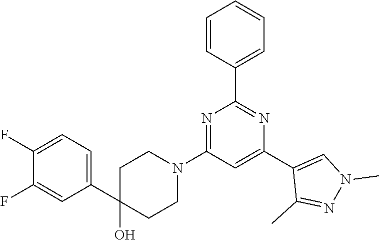 [SMILES: Cc1nn(C)cc1-c1cc(N2CCC(O)(c3ccc(F)c(F)c3)CC2)nc(-c2ccccc2)n1] | 4-(3,4-difluorophenyl)- 1-(6-(1,3-dimethyl-1H- pyrazol-4-yl)-2- phenylpyrimidin-4- yl)piperidin-4-ol | Calc'd 462.2, Found 462.4 |
| 60 | 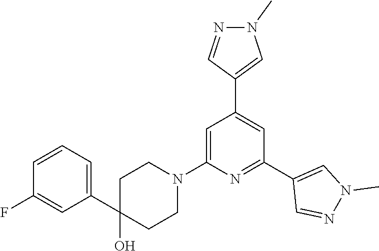 [SMILES: Cn1cc(-c2cc(-c3cnn(C)c3)nc(N3CCC(O)(c4cccc(F)c4)CC3)c2)cn1] | 1-(4,6-bis(1-methyl-1H- pyrazol-4-yl)pyridin-2- yl)-4-(3- fluorophenyl)piperidin- 4-ol | Calc'd 433.2, Found 433.2 |
| 61 | 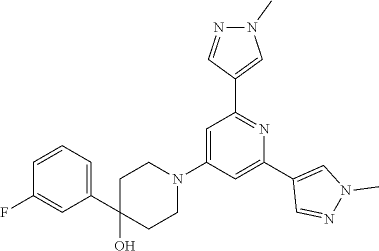 [SMILES: Cn1cc(-c2cc(N3CCC(O)(c4cccc(F)c4)CC3)cc(-c3cnn(C)c3)n2)cn1] | 1-(2,6-bis(1-methyl-1H- pyrazol-4-yl)pyridin-4- yl)-4-(3- fluorophenyl)piperidin- 4-ol | Calc'd 433.2, Found 433.2 |
| 62 | 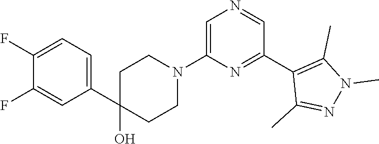 [SMILES: Cc1nn(C)c(C)c1-c1cncc(N2CCC(O)(c3ccc(F)c(F)c3)CC2)n1] | 4-(3,4-difluorophenyl)- 1-(6-(1,3,5-trimethyl- 1H-pyrazol-4- yl)pyrazin-2- yl)piperidin-4-ol | Calc'd 400.2, Found 400.2 |
| 63 | 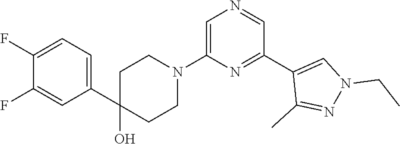 [SMILES: CCn1cc(-c2cncc(N3CCC(O)(c4ccc(F)c(F)c4)CC3)n2)c(C)n1] | 4-(3,4-difluorophenyl)- 1-(6-(1-ethyl-3-methyl- 1H-pyrazol-4- yl)pyrazin-2- yl)piperidin-4-ol | Calc'd 400.2, Found 400.2 |
| 87 | 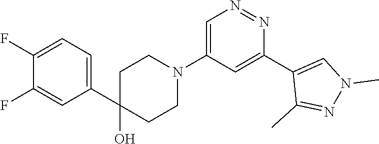 [SMILES: Cc1nn(C)cc1-c1cc(N2CCC(O)(c3ccc(F)c(F)c3)CC2)cnn1] | 4-(3,4-difluorophenyl)- 1-(6-(1,3-dimethyl-1H- pyrazol-4-yl)pyridazin- 4-yl)piperidin-4-ol | Calc'd 386.4, Found 386.2 |
| 88 | 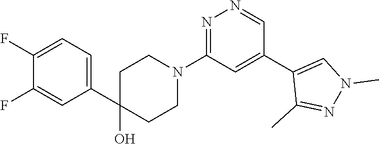 [SMILES: Cc1nn(C)cc1-c1cnnc(N2CCC(O)(c3ccc(F)c(F)c3)CC2)c1] | 4-(3,4-difluorophenyl)- 1-(5-(1,3-dimethyl-1H- pyrazol-4-yl)pyridazin- 3-yl)piperidin-4-ol | Calc'd 386.4, Found 386.3 |
| 89 | 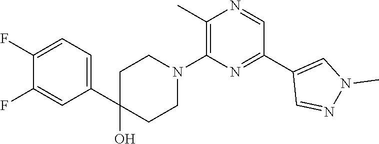 [SMILES: Cc1ncc(-c2cnn(C)c2)nc1N1CCC(O)(c2ccc(F)c(F)c2)CC1] | 4-(3,4-difluorophenyl)- 1-(3-methyl-6-(1- methyl-1H-pyrazol-4- yl)pyrazin-2- yl)piperidin-4-ol | Calc'd 386.4, Found 386.3 |
| 90 | 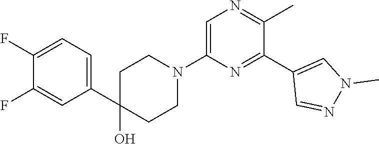 [SMILES: Cc1ncc(N2CCC(O)(c3ccc(F)c(F)c3)CC2)nc1-c1cnn(C)c1] | 4-(3,4-difluorophenyl)- 1-(5-methyl-6-(1- methyl-1H-pyrazol-4- yl)pyrazin-2- yl)piperidin-4-ol | Calc'd 386.4, Found 386.3 |
| 91 | 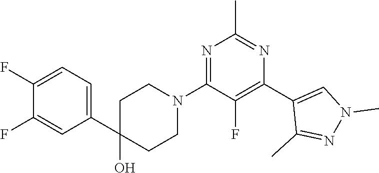 [SMILES: Cc1nc(-c2cn(C)nc2C)c(F)c(N2CCC(O)(c3ccc(F)c(F)c3)CC2)n1] | 4-(3,4-difluorophenyl)- 1-(6-(1,3-dimethyl-1H- pyrazol-4-yl)-5-fluoro-2- methylpyrimidin-4- yl)piperidin-4-ol | Calc'd 418.4, Found 418.4 |
| 92 | 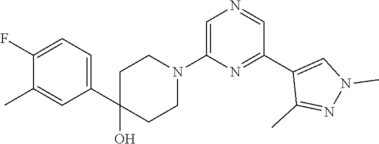 | 1-(6-(1,3-dimethyl-1H- pyrazol-4-yl)pyrazin-2- yl)-4-(4-fluoro-3- methylphenyl)piperidin- 4-ol | Calc'd 382.5, Found 382.4 |
| 93 | 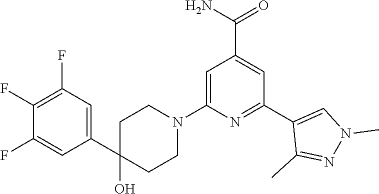 [SMILES: Cc1nn(C)cc1-c1cc(C(N)=O)cc(N2CCC(O)(c3cc(F)c(F)c(F)c3)CC2)n1] | 2-(1,3-dimethyl-1H- pyrazol-4-yl)-6-(4- hydroxy-4-(3,4,5- trifluorophenyl)piperidin- 1-yl)isonicotinamide | Calc'd 445.5, Found 445.4 |
| 94 | 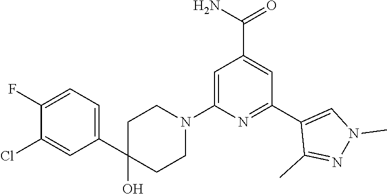 [SMILES: Cc1nn(C)cc1-c1cc(C(N)=O)cc(N2CCC(O)(c3ccc(F)c(Cl)c3)CC2)n1] | 2-(4-(3-chloro-4- fluorophenyl)-4- hydroxypiperidin-1-yl)- 6-(1,3-dimethyl-1H- pyrazol-4- yl)isonicotinamide | Calc'd 444.9, Found 444.3 |
| 95 | 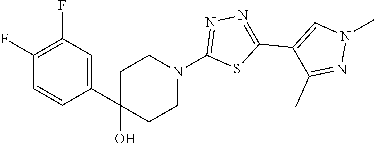 [SMILES: Cc1nn(C)cc1-c1nnc(N2CCC(O)(c3ccc(F)c(F)c3)CC2)s1] | 4-(3,4-difluorophenyl)- 1-(5-(1,3-dimethyl-1H- pyrazol-4-yl)-1,3,4- thiadiazol-2- yl)piperidin-4-ol | Calc'd 392.4, Found 392.2 |
| 96 | 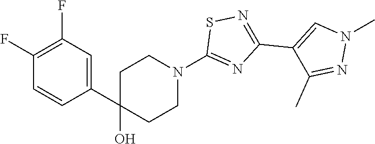 [SMILES: Cc1nn(C)cc1-c1nsc(N2CCC(O)(c3ccc(F)c(F)c3)CC2)n1] | 4-(3,4-difluorophenyl)- 1-(3-(1,3-dimethyl-1H- pyrazol-4-yl)-1,2,4- thiadiazol-5- yl)piperidin-4-ol | Calc'd 392.4, Found 392.4 |
| 97 | 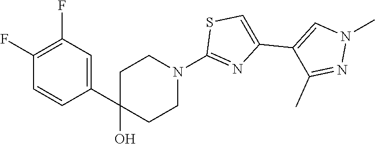 [SMILES: Cc1nn(C)cc1-c1csc(N2CCC(O)(c3ccc(F)c(F)c3)CC2)n1] | 4-(3,4-difluorophenyl)- 1-(4-(1,3-dimethyl-1H- pyrazol-4-yl)thiazol-2- yl)piperidin-4-ol | Calc'd 391.5, Found 391.5 |
| 98 | 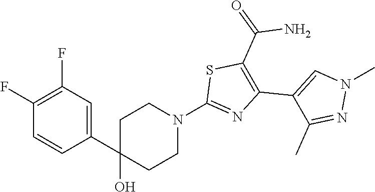 [SMILES: Cc1nn(C)cc1-c1nc(N2CCC(O)(c3ccc(F)c(F)c3)CC2)sc1C(N)=O] | 2-(4-(3,4- difluorophenyl)-4- hydroxypiperidin-1-yl)- 4-(1,3-dimethyl-1H- pyrazol-4-yl)thiazole-5- carboxamide | Calc'd 434.5, Found 434.4 |
| 99 | 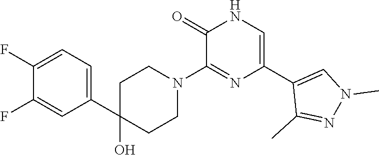 [SMILES: Cc1nn(C)cc1-c1c[nH]c(=O)c(N2CCC(O)(c3ccc(F)c(F)c3)CC2)n1] | 3-(4-(3,4- difluorophenyl)-4- hydroxypiperidin-1-yl)- 5-(1,3-dimethyl-1H- pyrazol-4-yl)pyrazin- 2(1H)-one | Calc'd 402.4, Found 402.4 |
| 100 | 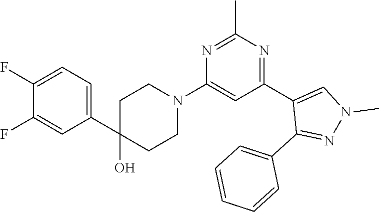 [SMILES: Cc1nc(-c2cn(C)nc2-c2ccccc2)cc(N2CCC(O)(c3ccc(F)c(F)c3)CC2)n1] | 4-(3,4-difluorophenyl)- 1-(2-methyl-6-(1- methyl-3-phenyl-1H- pyrazol-4-yl)pyrimidin- 4-yl)piperidin-4-ol | Calc'd 462.5, Found 462.4 |
| 101 | 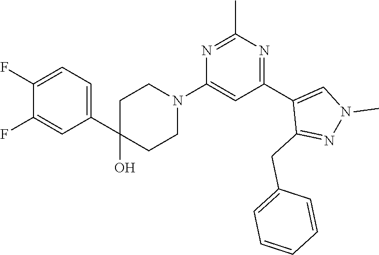 [SMILES: Cc1nc(-c2cn(C)nc2Cc2ccccc2)cc(N2CCC(O)(c3ccc(F)c(F)c3)CC2)n1] | 1-(6-(3-benzyl-1- methyl-1H-pyrazol-4- yl)-2-methylpyrimidin- 4-yl)-4-(3,4- difluorophenyl)piperidin- 4-ol | Calc'd 476.5, Found 476.5 |
| 102 | 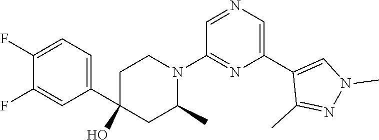 [SMILES: Cc1nn(C)cc1-c1cncc(N2CC[C@@](O)(c3ccc(F)c(F)c3)C[C@@H]2C)n1] | (rac)-cis-4-(3,4- difluorophenyl)-1-(6- (1,3-dimethyl-1H- pyrazol-4-yl)pyrazin-2- yl)-2-methylpiperidin-4- ol | Calc'd 400.5, Found 400.4 |
| 103 | 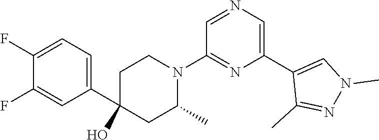 [SMILES: Cc1nn(C)cc1-c1cncc(N2CC[C@@](O)(c3ccc(F)c(F)c3)C[C@H]2C)n1] | (rac)-trans-4-(3,4- difluorophenyl)-1-(6- (1,3-dimethyl-1H- pyrazol-4-yl)pyrazin-2- yl)-2-methylpiperidin-4- ol | Calc'd 400.5, Found 400.4 |

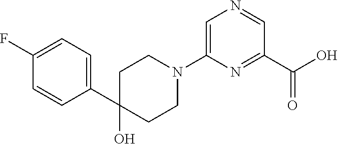 [SMILES: O=C(O)c1cncc(N2CCC(O)(c3ccc(F)cc3)CC2)n1]

### Method 5. step 1. 6-(4-(4-fluorophenyl)-4-hydroxypiperidin-1-yl)pyrazine-2-carboxylic acid

6-chloropyrazine-2-carboxylic acid (632 mg, 3.98 mmol), 4-(4-fluorophenyl)piperidin-4-ol (1.16 g, 5.97 mmol), and potassium carbonate (1.09 g, 7.96 mmol) were combined in DMA (6 mL) and stirred at 80° C. overnight. The mixture was filtered through a fritted funnel and the solution was directly purified by reverse phase chromatography (Biotage 60 g C₁₈ cartridge; 5-40% ACN in water+0.1% TFA) to afford the title compound (675 mg, 39%) as the trifluoroacetic acid salt. LCMS: m/z=318.1 [M+1].

### Example 64

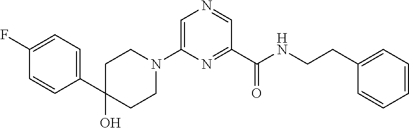 [SMILES: O=C(NCCc1ccccc1)c1cncc(N2CCC(O)(c3ccc(F)cc3)CC2)n1]

### 6-(4-(4-fluorophenyl)-4-hydroxypiperidin-1-yl)-N-phenethylpyrazine-Z-carboxamide

### Method 5, Step 2. 6-(4-(4-fluorophenyl)-4-hydroxypiperidin-1-yl)-N-phenethylpyrazine-2-carboxamide

6-[4-(4-fluorophenyl)-4-hydroxypiperidin-1-yl]pyrazine-2-carboxylic acid (40 mg, 126 μmol) and HATU (52.4 mg, 138 μmol) were dissolved in DMF (1 mL) and allowed to stir for 15 minutes. Then 1-(pyridin-2-yl)methanamine (40.8 mg, 378 μmol) and triethylamine (87.7 μL, 630 μmol) were added and the reaction mixture was stirred overnight. The reaction mixture was directly purified by preparative HPLC to attain 6-[4-(4-fluorophenyl)-4-hydroxypiperidin-1-yl]-N-[(pyridin-2-yl)methyl]pyrazine-2-carboxamide (37 mg, 72%). ¹H NMR (400 MHz, DMSO-d6) δ=8.56-8.50 (m, 2H), 8.29 (s, 1H), 7.55-7.49 (m, 2H), 7.29-7.08 (m, 7H), 5.22 (s, 1H), 4.38 (m, 2H), 3.54-3.45 (m, 2H), 3.35-3.23 (m, 2H), 2.83 (t, J=7.6 Hz, 2H), 1.93 (dt, J=4.4, 13.0 Hz, 2H), 1.68 (m, 2H). LCMS: m/z=421.2 [M+1].

The compounds in Table 3 were prepared using similar procedures to those described for Example 65 using the appropriate starting materials.

TABLE 3

| | | | Exact |
|---|---|---|---|
| Ex | | | Mass |
| Nos. | Structure | IUPAC Name | [M + 1] |
| 65 | 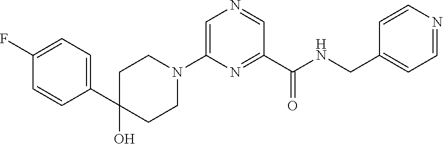 [SMILES: O=C(NCc1ccncc1)c1cncc(N2CCC(O)(c3ccc(F)cc3)CC2)n1] | 6-(4-(4-fluorophenyl)-4- hydroxypiperidin-1-yl)- N-(pyridin-4- ylmethyl)pyrazine-2- carboxamide | Calc'd 408.2, Found 408.2 |
| 66 | 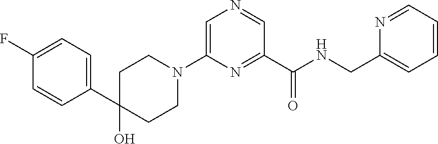 [SMILES: O=C(NCc1ccccn1)c1cncc(N2CCC(O)(c3ccc(F)cc3)CC2)n1] | 6-(4-(4-fluorophenyl)-4- hydroxypiperidin-1-yl)- N-(pyridin-2- ylmethyl)pyrazine-2- carboxamide | Calc'd 408.2, Found 408.2 |
| 67 | 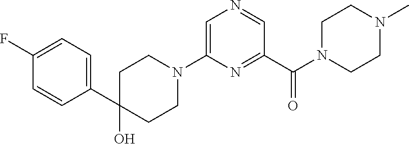 [SMILES: CN1CCN(C(=O)c2cncc(N3CCC(O)(c4ccc(F)cc4)CC3)n2)CC1] | (6-(4-(4-fluorophenyl)- 4-hydroxypiperidin-1- yl)pyrazin-2-yl)(4- methylpiperazin-1- yl)methanone | Calc'd 400.2, Found 400.2 |
| 68 | 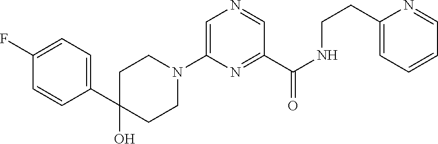 [SMILES: O=C(NCCc1ccccn1)c1cncc(N2CCC(O)(c3ccc(F)cc3)CC2)n1] | 6-(4-(4-fluorophenyl)-4- hydroxypiperidin-1-yl)- N-(2-(pyridin-2- yl)ethyl)pyrazine-2- carboxamide | Calc'd 422.2, Found 422.2 |
| 69 | 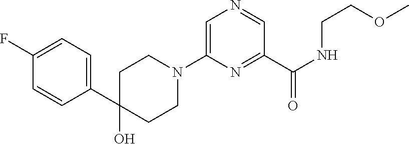 [SMILES: COCCNC(=O)c1cncc(N2CCC(O)(c3ccc(F)cc3)CC2)n1] | 6-(4-(4-fluorophenyl)-4- hydroxypiperidin-1-yl)- N-(2- methoxyethyl)pyrazine- 2-carboxamide | Calc'd 375.2, Found 396.2 [M + Na] |
| 70 | 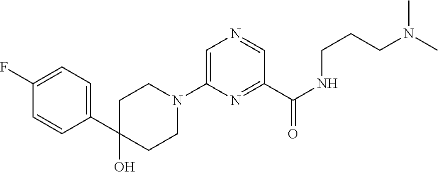 [SMILES: CN(C)CCCNC(=O)c1cncc(N2CCC(O)(c3ccc(F)cc3)CC2)n1] | N-(3- (dimethylamino)propyl)- 6-(4-(4-fluorophenyl)-4- hydroxypiperidin-1- yl)pyrazine-2- carboxamide | Calc'd 402.2, Found 402.2 |
| 71 | 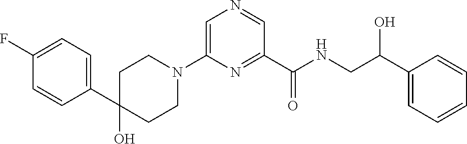 [SMILES: O=C(NCC(O)c1ccccc1)c1cncc(N2CCC(O)(c3ccc(F)cc3)CC2)n1] | 6-(4-(4-fluorophenyl)-4- hydroxypiperidin-1-yl)- N-(2-hydroxy-2- phenylethyl)pyrazine-2- carboxamide | Calc'd 437.2, Found 437.2 |

### Example 72

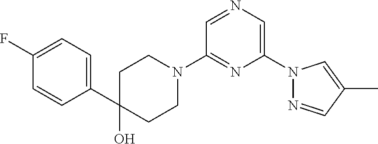 [SMILES: Cc1cnn(-c2cncc(N3CCC(O)(c4ccc(F)cc4)CC3)n2)c1]

### 4-(4-fluorophenyl)-1-(6-(4-methyl-1H-pyrazol-1-yl)pyrazin-2-yl)piperidin-4-ol

### Method 6. 4-(4-fluorophenyl)-1-(6-(4-methyl-1H-pyrazol-1-yl)pyrazin-2-yl)piperidin-4-ol

1-(6-chloropyrazin-2-yl)-4-(4-fluorophenyl)piperidin-4-ol (31 mg, 100 μmol), 4-methyl-1H-pyrazole (49.2 mg, 600 μmol), and cesium carbonate (64.9 mg, 200 mol) were combined in DMA (300 μL) and heated to 90° C. overnight. The reaction was diluted with ethyl acetate and the organic layer was washed with brine three times. The organic extract was dried over Na₂SO₄, filtered, and concentrated. The residue was purified by silica gel chromatography (Biotage 10 g silica cartridge; 0-60% ethyl acetate in heptanes) to afford the title compound. ¹H NMR (400 MHz, DMSO-d6) δ=8.36 (s, 1H), 8.24 (d, J=8.3 Hz, 2H), 7.62 (s, 1H), 7.57-7.44 (m, 2H), 7.10 (t, J=8.8 Hz, 2H), 5.23 (s, 1H), 4.34 (m, 2H), 3.35-3.27 (m, 2H), 2.09 (s, 3H), 1.96 (dt, J=4.4, 13.0 Hz, 2H), 1.70 (m, 2H). LCMS: m/z=354.2 [M+1].

### Example 73

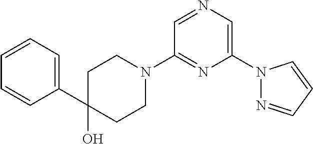 [SMILES: OC1(c2ccccc2)CCN(c2cncc(-n3cccn3)n2)CC1]

1-(6-(1H-pyrazol-1-yl)pyrazin-2-yl)-4-phenylpiperidin-4-ol

### Method 7. 1-(6-(1H-pyrazol-1-yl)pyrazin-2-yl)-4-phenylpiperidin-4-ol

4-phenylpiperidin-4-ol (42.1 mg, 238 μmol), 2-chloro-6-(1H-pyrazol-1-yl)pyrazine (36 mg, 199 μmol), and potassium carbonate (55.0 mg, 398 μmol) were combined in DMA (500 μL) and heated to 90° C. for 1 h. The reaction was cooled to RT and directly purified by reverse phase chromatography (Biotage 30 g C₁₈ cartridge; 5-90% ACN in water+0.1% TFA). The fractions containing product were concentrated. The residue was taken up in DCM and the organic layer was washed with saturated aqueous NaHCO₃ solution. The organic layer was then dried over Na₂SO₄, filtered and concentrated to afford the title compound (23.7 mg, 37%). LCMS: m/z=322.2 [M+1].

The compounds in Table 4 were prepared using similar procedures to those described for Example 73 using the appropriate starting materials.

TABLE 4

| | | | Exact |
|---|---|---|---|
| Ex | | | Mass |
| Nos. | Structure | IUPAC Name | [M + 1] |
| 74 | 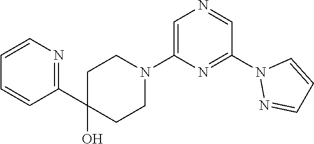 [SMILES: OC1(c2ccccn2)CCN(c2cncc(-n3cccn3)n2)CC1] | 1-(6-(1H-pyrazol-1- yl)pyrazin-2-yl)-4- (pyridin-2-yl)piperidin- 4-ol | Calc'd 323.2, Found 323.3 |
| 75 | 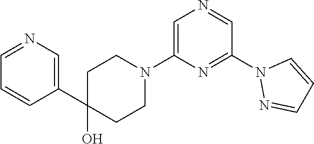 [SMILES: OC1(c2cccnc2)CCN(c2cncc(-n3cccn3)n2)CC1] | 1-(6-(1H-pyrazol-1- yl)pyrazin-2-yl)-4- (pyridin-3-yl)piperidin- 4-ol | Calc'd 323.2, Found 323.2 |
| 76 | 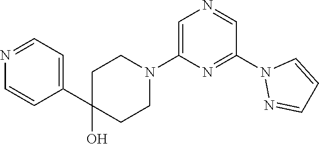 [SMILES: OC1(c2ccncc2)CCN(c2cncc(-n3cccn3)n2)CC1] | 1-(6-(1H-pyrazol-1- yl)pyrazin-2-yl)-4- (pyridin-4-yl)piperidin- 4-ol | Calc'd 323.2, Found 323.2 |
| 77 | 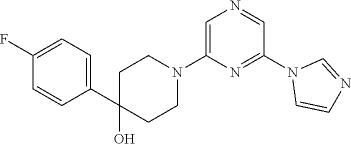 [SMILES: OC1(c2ccc(F)cc2)CCN(c2cncc(-n3ccnc3)n2)CC1] | 1-(6-(1H-pyrazol-1- yl)pyrazin-2-yl)-4-(4- fluorophenyl)piperidin- 4-ol | Calc'd 340.2, Found 340.2 |
| 78 | 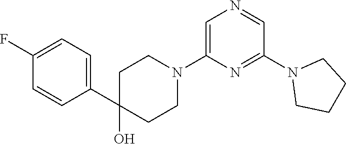 [SMILES: OC1(c2ccc(F)cc2)CCN(c2cncc(N3CCCC3)n2)CC1] | 4-(4-fluorophenyl)-1-(6- (pyrrolidin-1-yl)pyrazin- 2-yl)piperidin-4-ol | Calc'd 343.2, Found 343.2 |
| 79 | 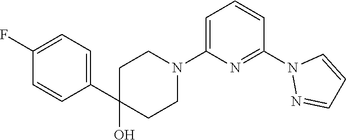 [SMILES: OC1(c2ccc(F)cc2)CCN(c2cccc(-n3cccn3)n2)CC1] | 1-(6-(1H-pyrazol-1- yl)pyridin-2-yl)-4-(4- fluorophenyl)piperidin- 4-ol | Calc'd 339.2, Found 339.2 |
| 80 | 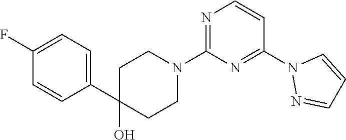 [SMILES: OC1(c2ccc(F)cc2)CCN(c2nccc(-n3cccn3)n2)CC1] | 1-(4-(1H-pyrazol-1- yl)pyrimidin-2-yl)-4-(4- fluorophenyl)piperidin- 4-ol | Calc'd 340.2, Found 340.2 |
| 81 | 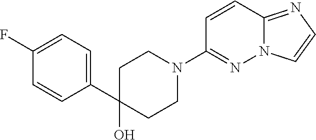 [SMILES: OC1(c2ccc(F)cc2)CCN(c2ccc3nccn3n2)CC1] | 4-(4-fluorophenyl)-1- (imidazo[1,2- b]pyridazin-6- yl)piperidin-4-ol | Calc'd 313.2, Found 313.2 |
| 82 | 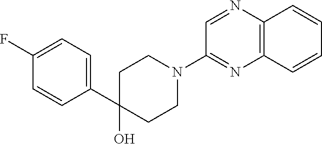 [SMILES: OC1(c2ccc(F)cc2)CCN(c2cnc3ccccc3n2)CC1] | 4-(4-fluorophenyl)-1- (quinoxalin-2- yl)piperidin-4-ol | Calc'd 324.1, Found 324.2 |
| 83 | 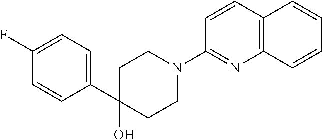 [SMILES: OC1(c2ccc(F)cc2)CCN(c2ccc3ccccc3n2)CC1] | 4-(4-fluorophenyl)-1- (quinolin-2-yl)piperidin- 4-ol | Calc'd 323.2, Found 323.2 |
| 84 | 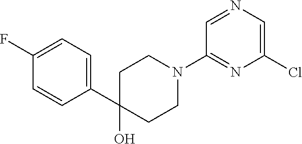 [SMILES: OC1(c2ccc(F)cc2)CCN(c2cncc(Cl)n2)CC1] | 1-(6-chloropyrazin-2- yl)-4-(4- fluorophenyl)piperidin- 4-ol | Calc'd 308.1, Found 308.1 |
| 85 | 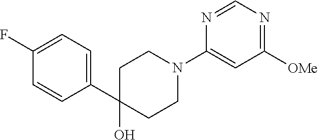 [SMILES: COc1cc(N2CCC(O)(c3ccc(F)cc3)CC2)ncn1] | 4-(4-fluorophenyl)-1-(6- methoxypyrimidin-4- yl)piperidin-4-ol | Calc'd 304.1, Found 304.1 |
| 86 | 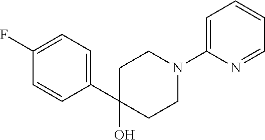 [SMILES: OC1(c2ccc(F)cc2)CCN(c2ccccn2)CC1] | 4-(4-fluorophenyl)-1- (pyridin-2-yl)piperidin- 4-ol | Calc'd 273.1, Found 273.1 |
| 104 | 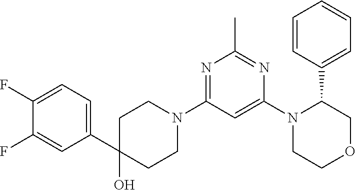 [SMILES: Cc1nc(N2CCC(O)(c3ccc(F)c(F)c3)CC2)cc(N2CCOC[C@H]2c2ccccc2)n1] | (R)-4-(3,4- difluorophenyl)-1-(2- methyl-6-(3- phenylmorpholino) pyrimidin-4-yl)piperidin- 4-ol | Calc'd 467.5, Found 467.4 |
| 105 | 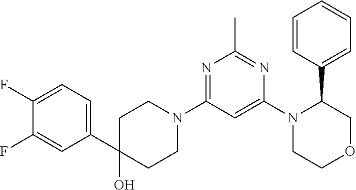 [SMILES: Cc1nc(N2CCC(O)(c3ccc(F)c(F)c3)CC2)cc(N2CCOC[C@@H]2c2ccccc2)n1] | (S)-4-(3,4- difluorophenyl)-1-(2- methyl-6-(3- phenylmorpholino) pyrimidin-4-yl)piperidin- 4-ol | Calc'd 467.5, Found 467.4 |
| 106 | 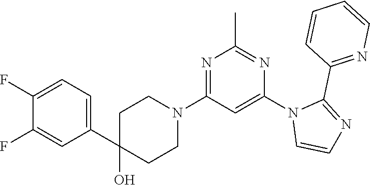 [SMILES: Cc1nc(N2CCC(O)(c3ccc(F)c(F)c3)CC2)cc(-n2ccnc2-c2ccccn2)n1] | 4-(3,4-difluorophenyl)- 1-(2-methyl-6-(2- (pyridin-2-yl)-1H- imidazol-1-yl)pyrimidin- 4-yl)piperidin-4-ol | Calc'd 449.5, Found 449.5 |
| 107 | 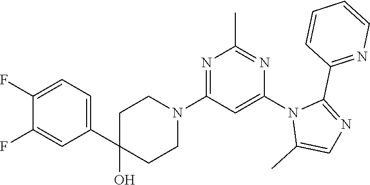 [SMILES: Cc1nc(N2CCC(O)(c3ccc(F)c(F)c3)CC2)cc(-n2c(C)cnc2-c2ccccn2)n1] | 4-(3,4-difluorophenyl)- 1-(2-methyl-6-(5- methyl-2-(pyridin-2-yl)- 1H-imidazol-1- yl)pyrimidin-4- yl)piperidin-4-ol | Calc'd 463.5, Found 463.4 |
| 108 | 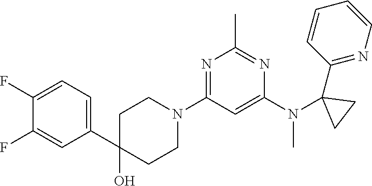 [SMILES: Cc1nc(N2CCC(O)(c3ccc(F)c(F)c3)CC2)cc(N(C)C2(c3ccccn3)CC2)n1] | 4-(3,4-difluorophenyl)- 1-(2-methyl-6- (methyl(1-(pyridin-2- yl)cyclopropyl)amino) pyrimidin-4-yl) piperidin-4-ol | Calc'd 452.5, Found 452.4 |

### Biochemical Assays

1. Silencing TREX1 in Tumor Cells

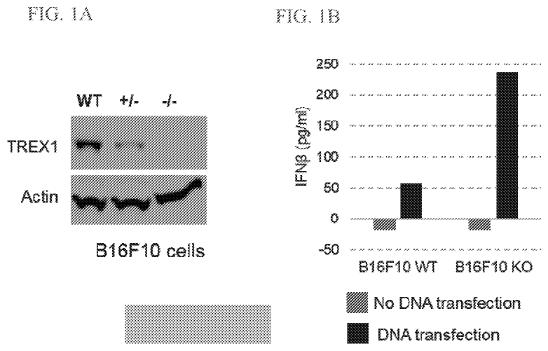

Activation of the cGAS/STING pathway upon sensing of cytosolic DNA and subsequent type I IFN production can occur in both tumor cells and innate immune cells, particularly dendritic cells. To evaluate whether TREX1 keeps in check the production of type I IFN by a well described, cold syngeneic tumor model that undergoes immune-mediated rejection upon activation of type I IFN by STING agonists, TREX1 was knocked down in B16F10 tumor cells using CRISPR (FIG. 1A). Accumulation of cytosolic DNA via DNA transfection of the tumor cells resulted in an about 5-fold increase in IFNβ production by the TREX1 knockout B16F10 cells relative to the parental tumor cells, demonstrating that TREX1 attenuated the activation of the cGAS/STING pathway in B16F10 tumor cells (FIG. 1B).

2. Growth of TREX1-Competent and -Deficient B16F10 Tumor Cells In Vivo

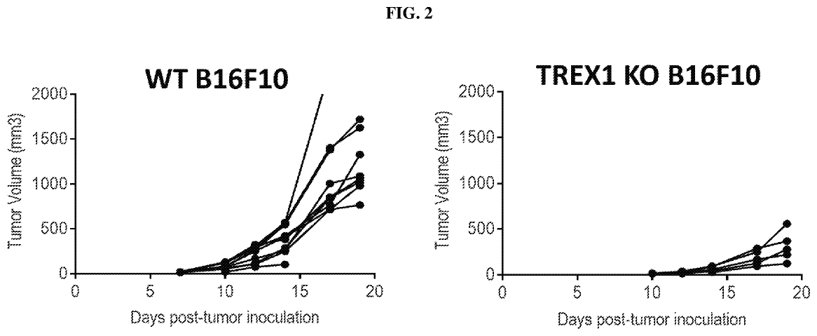

The growth of TREX1-competent and -deficient B16F10 tumor cells in vivo was evaluated. C57BL/6J mice were inoculated subcutaneously on the right flank with 300,000 parental or TREX1 knockout B16F10 tumor cells. Body weights were collected two times per week, and tumor measurements, two to three times per week, starting when tumors became measurable and for the remaining duration of the study. Tumors in which TREX1 had been silenced presented with remarkably smaller volumes than the parental B16F10 tumors (FIG. 2).

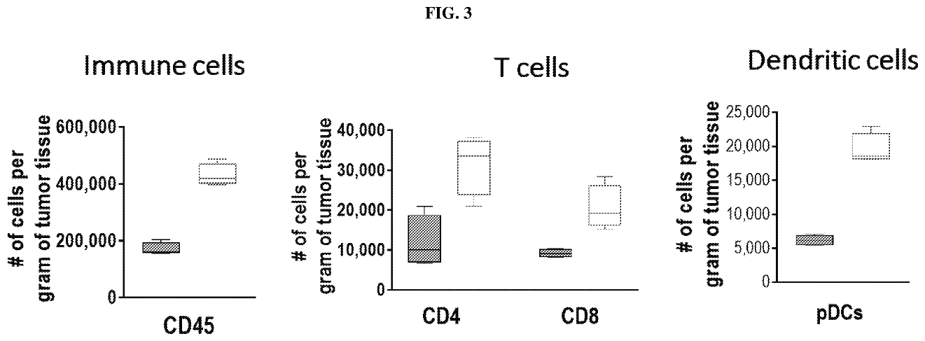

Tumors were harvested on day 19, upon termination of the study, and digested into single cell suspensions to enable flow cytometry quantification of tumor-infiltrating immune populations. TREX1 knockout B16F10 tumors were found to exhibit a significant increase in overall immune cells, which reflected an increase in the number of tumor infiltrating CD4 and CD8 T cells as well as in plasmacytoid dendritic cells (pDCs) (FIG. 3). pDCs are known to play a central role in the induction of antigen-specific anti-tumor immune responses whereas T cells are known to be major effectors of anti-tumor efficacy in mice and humans. The profound change in the immune infiltrate of the tumors deficient in TREX1 thus suggest that the inhibition of the growth of the latter tumors is at least in part immune-mediated.

### TREX1 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged full length human TREX1 (expressed in E. coli and purified in house) in reaction buffer (50 mM Tris (pH 7.4), 150 mM NaCl, 2 mM DTT, 0.1 mg/mL BSA, 0.01% (v/v) Tween-20 and either 100 mM MgCl₂) was added to a 384-well Black ProxiPlate Plus (Perkin Elmer) which already contained compound (150 nL) at varying concentrations as a 10 point dose-response in DMSO. To this was added 7.5 μL of dsDNA substrate (Strand A: 5′ TEX615/GCT AGG CAG 3′; Strand B: 5′ CTG CCT AGC/IAbRQSp (Integrated DNA Technologies)) in reaction buffer. Final concentrations were 150 pM TREX1, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction were 112.5 pM TREX1, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

### TREX2 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged human TREX2 (residues M44-A279, expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10 point dose-response in DMSO. To this was added 7.5 μL of dsDNA substrate (Strand A: 5′ TEX615/GCT AGG CAG 3′; Strand B: 5′ CTG CCT AGC/IAbRQSp (IDT)) in reaction buffer. Final concentrations were 2.5 nM TREX2, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction mixture were 1.875 pM TREX2, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

TABLE 5
Results are shown in Table 5.

| | TREX1 | TREX2 |
|---|---|---|
| Example | IC₅₀ (μM) | IC₅₀ (μM) |
| 1 | C | C |
| 2 | D | D |
| 3 | D | D |
| 4 | B | C |
| 5 | D | D |
| 6 | D | D |
| 7 | C | D |
| 8 | B | D |
| 9 | A | C |
| 10 | A | C |
| 11 | B | C |
| 12 | C | D |
| 13 | C | D |
| 14 | C | D |
| 15 | B | D |
| 16 | C | D |
| 17 | B | C |
| 18 | C | D |
| 19 | C | D |
| 20 | C | D |
| 21 | C | D |
| 22 | C | D |
| 23 | C | D |
| 24 | D | D |
| 25 | C | D |
| 26 | C | D |
| 27 | D | D |
| 28 | D | D |
| 29 | D | D |
| 30 | C | D |
| 31 | C | D |
| 32 | D | D |
| 33 | D | D |
| 34 | C | D |
| 35 | C | D |
| 36 | B | D |
| 37 | C | D |
| 38 | C | D |
| 39 | A | C |
| 40 | B | D |
| 41 | A | C |
| 42 | C | D |
| 43 | B | D |
| 44 | B | C |
| 45 | A | C |
| 46 | B | D |
| 47 | A | C |
| 48 | A | C |
| 49 | C | D |
| 50 | D | D |
| 51 | B | D |
| 52 | C | D |
| 53 | C | D |
| 54 | C | D |
| 55 | D | D |
| 56 | B | D |
| 57 | A | D |
| 58 | A | C |
| 59 | C | D |
| 60 | C | D |
| 61 | C | D |
| 62 | A | C |
| 63 | A | B |
| 64 | C | D |
| 65 | D | D |
| 66 | D | D |
| 67 | D | D |
| 68 | C | D |
| 69 | D | D |
| 70 | D | D |
| 71 | C | D |
| 72 | C | D |
| 73 | D | D |
| 74 | D | D |
| 75 | D | D |
| 76 | D | D |
| 77 | C | D |
| 78 | D | D |
| 79 | C | D |
| 80 | C | D |
| 81 | C | D |
| 82 | C | D |
| 83 | D | D |
| 84 | C | D |
| 85 | C | D |
| 86 | D | D |
| 87 | C | D |
| 88 | B | D |
| 89 | C | D |
| 90 | B | D |
| 91 | B | D |
| 92 | B | D |
| 93 | B | D |
| 94 | B | D |
| 95 | C | D |
| 96 | B | D |
| 97 | C | D |
| 98 | B | D |
| 99 | B | D |
| 100 | B | D |
| 101 | B | D |
| 102 | C | D |
| 103 | C | D |
| 104 | C | D |
| 105 | C | D |
| 106 | C | D |
| 107 | C | D |
| 108 | B | D |

TREX1 IC₅₀: A =< 0.1 μM; B = 0.1 to 1 μM; C = 1 to 10 μM; D => 10 μM.
TREX2 IC₅₀: A =< 1 μM, B = 1 to 10 μM, C = 10 to 100 μM, D => 100 μM.

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 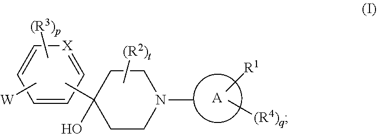
or a pharmaceutically acceptable salt thereof, wherein:
W is fluoro substituted meta or para to the piperidine;
X is independently N or C;
Ring A is a 5-membered heteroaryl or a 6-membered heteroaryl, wherein said 6-membered heteroaryl is substituted meta to the piperidine by R¹;
R¹ is phenyl, heteroaryl, heterocyclyl, C₁-C₆ alkoxy, C₁-C₆ alkyl, C₁-C₆ haloalkyl, hydroxyC₁-C₆ alkyl, —C(O)NR^{a}R^{b}, —NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OC₁-C₆ alkyl, or —SC₁-C₆ alkyl, wherein each of said phenyl, heteroaryl, and heterocyclyl are optionally substituted with 1 to 4 groups selected from R⁶;
R² is halo, hydroxy, C₁-C₆ alkyl, C₁-C₆ haloalkyl, hydroxyC₁-C₆ alkyl, C₁-C₆ alkoxy, C₁-C₆ haloalkoxy, —C(O)NR^{a}R^{b}, —NR^{a}R^{b}, —COOR^{c}, —SO₂R^{c}, —NR^{a}C(O)OR^{c}, —NR^{a}C(S)OR^{c}, or —NR^{a}C(O)R^{c};
each R³, if present, is independently halo, hydroxy, C₁-C₆ alkyl, C₁-C₆ haloalkyl, hydroxyC₁-C₆ alkyl, C₁-C₆ alkoxy, or C₁-C₆ haloalkoxy;
R⁴ is heteroaryl, halo, C₁-C₆ alkoxy, C₁-C₆ alkyl, C₁-C₆ haloalkyl, hydroxyC₁-C₆ alkyl, oxo, —C(O)NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OR^{c}, or —SR^{c}, wherein said heteroaryl is optionally substituted with 1 to 3 groups selected from R⁵;
R⁵ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c}
R⁶ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c};
each R^{a} is independently hydrogen or C₁-C₆ alkyl;
each R^{b} is independently hydrogen or C₁-C₆ alkyl optionally substituted with 1 or 2 groups selected from phenyl, heteroaryl, OR^{c}, and —NR^{c}R^{d}; or R^{a} and R^{b} together with the nitrogen atom to which they are attached form a nitrogen containing heterocyclyl optionally substituted with 1 to 4 groups selected from halo, C₁-C₆ alkyl, C₁-C₆ haloalkyl, C₁-C₆ alkoxy, and C₁-C₆ haloalkoxy;
each R^{c} and R^{d} are independently hydrogen or C₁-C₆ alkyl;
p is 0, 1, or 2;
t is 0, 1, or 2; and
q is 0, 1, or 2;
provided the compound of Formula I is not 1-(2-amino-6-methylpyrimidin-4-yl)-4-(4-fluorophenyl)piperidin-4-ol, (R)-4-(4-fluorophenyl)-1-(6-((2-hydroxy-2-phenylethyl)amino)pyrimidin-4-yl)piperidin-4-ol, 4-(4-fluorophenyl)-1-(4-(1,3,5-trimethyl-1H-pyrazol-4-yl)pyrimidin-2-yl)piperidin-4-ol, 4-(4-fluorophenyl)-1-(2-methyl-6-(piperidin-3-yl)pyrimidin-4-yl)piperidin-4-ol, 4-(4-fluorophenyl)-1-(2,5,6-trimethylpyrimidin-4-yl)piperidin-4-ol, 1-(2-amino-5-ethylpyrimidin-4-yl)-4-(4-fluorophenyl)piperidin-4-ol, 4-(4-fluorophenyl)-1-(4-methylpyrimidin-2-yl)piperidin-4-ol, 4-(4-fluorophenyl)-1-(4-(pyridin-3-yl)pyrimidin-2-yl)piperidin-4-ol, 4-(4-fluorophenyl)-1-(4-(pyridin-2-yl)pyrimidin-2-yl)piperidin-4-ol, 4-(4-fluorophenyl)-1-(4-(pyridin-2-yl)pyrimidin-2-yl)piperidin-4-ol, 4-(4-fluorophenyl)-1-(4-methoxy-6-methylpyrimidin-2-yl)piperidin-4-ol, or 4-(4-fluorophenyl)-1-(4-methyl-6-morpholinopyrimidin-2-yl)piperidin-4-ol, or a pharmaceutically acceptable salt of any of the foregoing.

2. A pharmaceutical composition comprising
1) a compound having the Formula I: 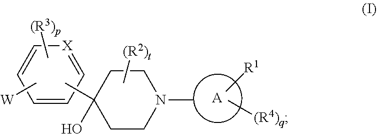
or a pharmaceutically acceptable salt thereof, wherein:
W is fluoro substituted meta or para to the piperidine;
X is independently N or C;
Ring A is a 5-membered heteroaryl or a 6-membered heteroaryl, wherein said 6-membered heteroaryl is substituted meta to the piperidine by R¹;
R¹ is phenyl, heteroaryl, heterocyclyl, C₁-C₆ alkoxy, C₁-C₆ alkyl, C₁-C₆ haloalkyl, hydroxyC₁-C₆ alkyl, —C(O)NR^{a}R^{b}, —NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OC₁-C₆ alkyl, or —SC₁-C₆ alkyl, wherein each of said phenyl, heteroaryl, and heterocyclyl are optionally substituted with 1 to 4 groups selected from R⁶;
R² is halo, hydroxy, C₁-C₆ alkyl, C₁-C₆ haloalkyl, hydroxyC₁-C₆ alkyl, C₁-C₆ alkoxy, C₁-C₆ haloalkoxy, —C(O)NR^{a}R^{b}, —NR^{a}R^{b}, —COOR^{c}, —SO₂R^{c}, —NR^{a}C(O)OR^{c}, —NR^{a}C(S)OR^{c}, or —NR^{a}C(O)R^{c};
each R³, if present, is independently halo, hydroxy, C₁-C₆ alkyl, C₁-C₆ haloalkyl, hydroxyC₁-C₆ alkyl, C₁-C₆ alkoxy, or C₁-C₆ haloalkoxy;
R⁴ is heteroaryl, halo, C₁-C₆ alkoxy, C₁-C₆ alkyl, C₁-C₆ haloalkyl, hydroxyC₁-C₆ alkyl, oxo, —C(O)NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OR^{c}, or —SR^{c}, wherein said heteroaryl is optionally substituted with 1 to 3 groups selected from R⁵;
R⁵ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c}
R⁶ is selected from halo, C₁-C₆ alkyl, C₁-C₆ haloalkyl, C₁-C₆ alkoxy, C₁-C₆ haloalkoxy, (C₃-C₈)cycloalkyl, cyano, —C(O)NR^{a}R^{b}, —NR^{a}R^{b}, —SO₂R^{c}, —NR^{a}C(O)OR^{c}, —NR^{a}C(S)OR^{c}, —C(O)R^{c}, —C(S)R^{c}, —S(O)R^{c}, —C(O)OR^{c}, —C(S)OR^{c}, —C(S)NR^{a}R^{c}, —NR^{a}C(O)R^{c}, —NR^{a}C(S)R^{c}, —OR^{c}, and —SR^{c};
each R^{a} is independently hydrogen or C₁-C₆ alkyl;
each R^{b} is independently hydrogen or C₁-C₆ alkyl optionally substituted with 1 or 2 groups selected from phenyl, heteroaryl, OR^{c}, and —NR^{c}R^{d}; or R^{a} and R^{b} together with the nitrogen atom to which they are attached form a nitrogen containing heterocyclyl optionally substituted with 1 to 4 groups selected from halo, C₁-C₆ alkyl, C₁-C₆ haloalkyl, C₁-C₆ alkoxy, and C₁-C₆ haloalkoxy;
each R^{c} and R^{d} are independently hydrogen or C₁-C₆ alkyl;
p is 0, 1, or 2;
t is 0, 1, or 2; and
q is 0, 1, or 2; and
2) a pharmaceutically acceptable carrier.

3. The compound or composition of claim 1 or 2, wherein p is 1 or 2.

4. The compound or composition of any one of claims 1 to 3, wherein the compound is of the Formula II: 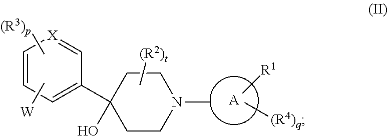
or a pharmaceutically acceptable salt thereof.

5. The compound or composition of any one of claims 1 to 4, wherein the compound is of the Formula III: 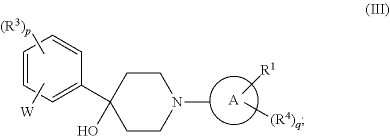
or a pharmaceutically acceptable salt thereof.

6. The compound or composition of any one of claims 1 to 5, wherein the compound is of the Formula IV: 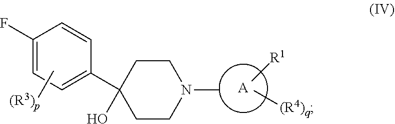
or a pharmaceutically acceptable salt thereof.

7. The compound or composition of any one of claims 1 to 6, wherein p is 1.

8. The compound or composition of any one of claims 1 to 7, wherein R³ is halo or C₁-C₆ alkyl.

9. The compound or composition of any one of claims 1 to 8, wherein R³ is methyl, fluoro or chloro.

10. The compound or composition of any one of claims 1 to 9, wherein Ring A is selected from: 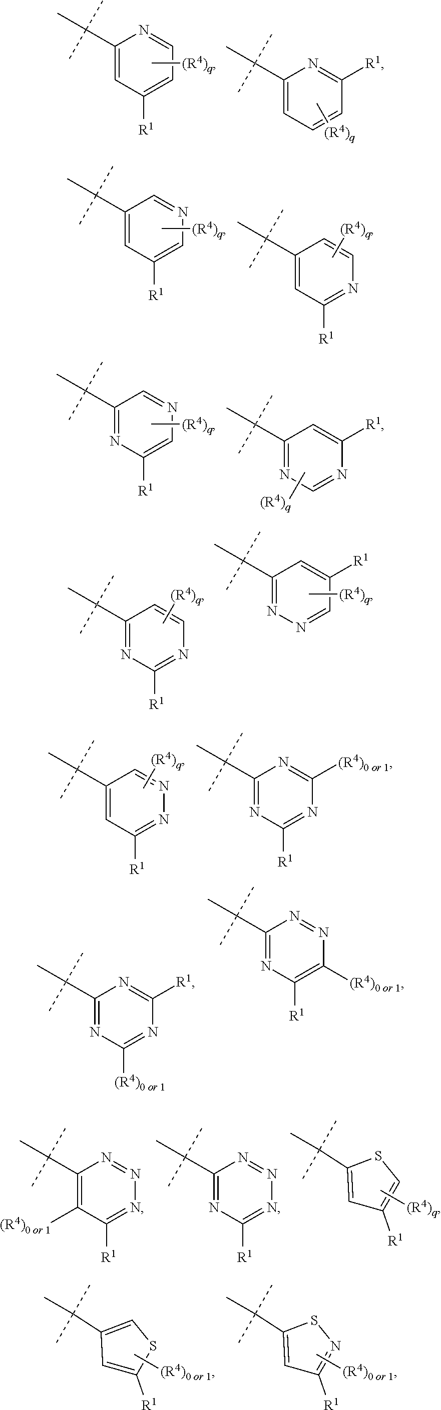 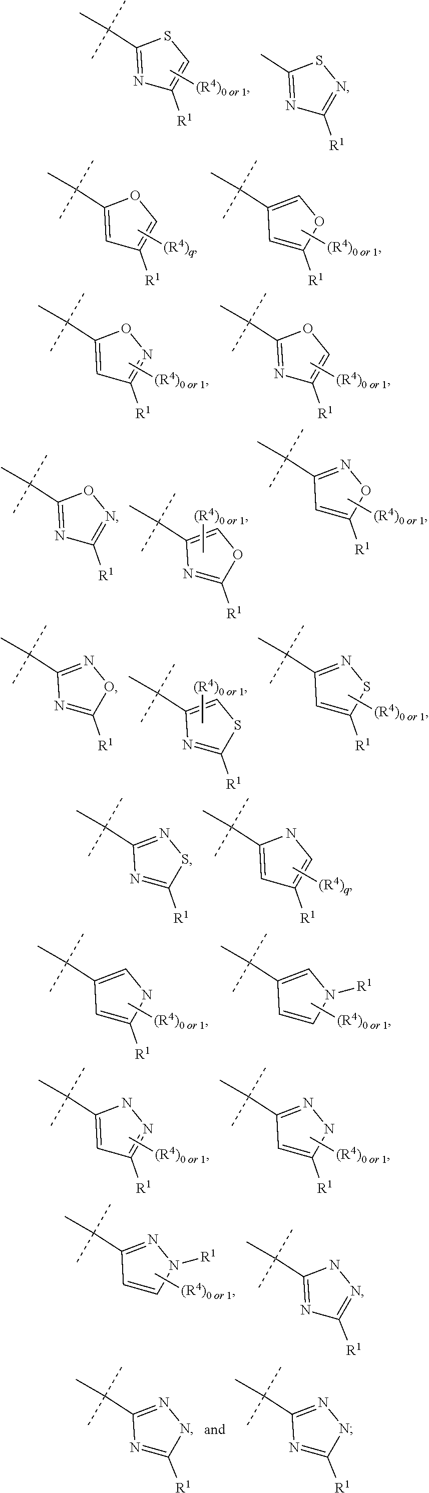
and
R¹ is phenyl, heteroaryl, heterocyclyl, C₁-C₆ alkoxy, C₁-C₆ alkyl, C₁-C₆ haloalkyl, hydroxyC₁-C₆ alkyl, —C(O)NR^{a}R^{b}, —COOR^{c}, —SO₂R^{c}, —NR^{a}C(O)OR^{c}, —NR^{a}C(S)OR^{c}, —C(O)R^{c}, —C(S)R^{c}, —S(O)R^{c}, —C(S)OR^{c}, —C(S)NR^{a}R^{c}, —NR^{a}C(O)R^{c}, —NR^{a}C(S)R^{c}, —OC₁-C₆ alkyl, or —SC₁-C₆ alkyl, wherein each of said phenyl, heteroaryl, and heterocyclyl are optionally substituted with 1 to 2 groups selected from R⁶.

11. The compound or composition of any one of claims 1 to 10, wherein ring A is

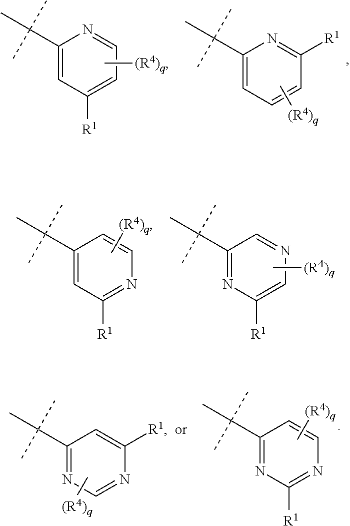

12. The compound or composition of any one of claims 1 to 11, wherein, q is 0 or 1.

13. The compound or composition of any one of claims 1 to 12, wherein R⁴ is 5- to 6-membered heteroaryl, —COOR^{c}, —C(O)NR^{a}R^{b}, C₁-C₆ alkyl, C₁-C₆ haloalkyl, hydroxyC₁-C₆ alkyl, wherein said 5- to 6-membered heteroaryl is optionally substituted with 1 or 2 groups selected from R⁵.

14. The compound or composition of any one of claims 1 to 13, wherein R⁴ is pyrazolyl, —COOR^{c}, —C(O)NR^{a}R^{b}, C₁-C₄ alkyl, C₁-C₄ haloalkyl, hydroxyC₁-C₄ alkyl, wherein said pyrazolyl is optionally substituted with 1 or 2 groups selected from R⁵.

15. The compound or composition of any one of claims 1 to 14, wherein R⁵ is C₁-C₄ alkyl.

16. The compound or composition of any one of claims 1 to 15, wherein R¹ is phenyl, heteroaryl, heterocyclyl, halo, C₁-C₆ alkoxy, C₁-C₆ alkyl, C₁-C₆ haloalkyl, hydroxyC₁-C₆ alkyl, —C(O)NR^{a}R^{b}, or —COOR^{c}, wherein each of said phenyl, heteroaryl, and heterocyclyl are optionally substituted with 1 to 3 groups selected from R⁶

17. The compound or composition of any one of claims 1 to 16, wherein R¹ is phenyl, 5- to 6-membered nitrogen containing heteroaryl, 5- to 6-membered nitrogen containing heterocyclyl, halo, C₁-C₃ alkoxy, C₁-C₃ alkyl, C₁-C₃ haloalkyl, oxo, —C(O)NR^{a}R^{b}, or —COOR^{c}, wherein each of said phenyl, heteroaryl, and heterocyclyl are optionally substituted with 1 to 3 groups selected from R⁶

18. The compound or composition of any one of claims 1 to 17, wherein R¹ is Cl, OCH₃, CH₃, CF₃, —C(CH₃)₂OR^{c}, —CH₂OR^{c}, CF₃, oxo, —COOR^{c}, or —C(O)NR^{a}R^{b}, phenyl, pyrazolyl, imidazolyl, isoxazolyl, triazolyl, pyridinyl, pyrimidinyl, or pyrrolidinyl, wherein each of said phenyl, pyrazolyl, imidazolyl, isoxazolyl, triazolyl, pyridinyl, pyrimidinyl, or pyrrolidinyl is optionally substituted with 1 to 3 groups selected from R⁶.

19. The compound or composition of any one of claims 1 to 18, wherein R⁶ is selected from halo, C₁-C₆ alkyl, C₁-C₆ haloalkyl, C₁-C₆ alkoxy, cycloalkyl, cyano, —C(O)NR^{a}R^{b}, and —SO₂R^{c}, wherein said C₁-C₆ alkyl is optionally substituted with phenyl.

20. The compound or composition of any one of claims 1 to 19, wherein R⁶ is selected from halo, C₁-C₃ alkyl, C₁-C₃ haloalkyl, C₁-C₃ alkoxy, 3- to 5-membered monocyclic cycloalkyl, cyano, —C(O)NR^{a}R^{b}, and —SO₂R^{c}, wherein said C₁-C₃ alkyl is optionally substituted with phenyl.

21. The compound or composition of any one of claims 1 to 20, wherein R⁶ is selected from F, CH₃, CF₃, CHF₂, OCH₃, cyclopropyl, cyano, benzyl, —C(O)NR^{a}R^{b}, or —SO₂R^{c}.

22. The compound or composition of any one of claims 1 to 21, wherein each R^{a} is independently hydrogen or CH₃.

23. The compound or composition of any one of claims 1 to 22, wherein each R^{b} is independently hydrogen or C₁-C₆ alkyl optionally substituted with 1 or 2 groups selected from phenyl, nitrogen containing heteroaryl, OR^{c}, or —NR^{c}R^{d}; or R^{a} and R^{b} together with the nitrogen atom to which they are attached form a nitrogen containing heterocyclyl optionally substituted with C₁-C₆ alkyl.

24. The compound or composition of any one of claims 1 to 23, wherein each R^{b} is independently hydrogen or C₁-C₃ alkyl optionally substituted with 1 or 2 groups selected from phenyl, 5- or 6-membered nitrogen containing heteroaryl, OR^{c}, or —NR^{c}R^{d}; or R^{a} and R^{b} together with the nitrogen atom to which they are attached form a 5- or 6-membered nitrogen containing heterocyclyl optionally substituted with C₁-C₃ alkyl.

25. The compound or composition of any one of claims 1 to 24, wherein each R^{b} is independently hydrogen or C₁-C₃ alkyl optionally substituted with 1 or 2 groups selected from phenyl, pyridinyl, OR^{c}, or —NR^{c}R^{d}; or R^{a} and R^{b} together with the nitrogen atom to which they are attached form a piperidinyl or piperazinyl optionally substituted with C₁-C₃ alkyl.

26. The compound or composition of any one of claims 1 to 25, wherein each R^{c} and R^{d} are independently hydrogen or CH₃.

27. A method of treating a disease responsive to the inhibition of TREX1 in a subject, comprising administering to the subject, a therapeutically effective amount of a compound of any one of claims 1 to 26, or a pharmaceutically acceptable salt thereof, or the composition of any one of claims 1 to 26.

28. The method of claim 27, wherein the disease is cancer.
